(19) United States
(12) Patent Application Publication
(10) Pub. No.: US 2026/0124215 A1
(43) Pub. Date: May 7, 2026

(54) METHODS OF TREATMENT OF INTESTINAL INFECTIONS

(71) Applicant: THE FRANCIS CRICK INSTITUTE LIMITED, London (GB)

(72) Inventors: Adam SATERIALE, London (GB); Noorul Bishara MARZOOK, London (GB)

(21) Appl. No.: 19/118,517

(22) PCT Filed: Oct. 4, 2023
(86) PCT No.: PCT/EP2023/077450

§ 371 Date: Apr. 4, 2025

(30) Foreign Application Priority Data
Oct. 4, 2022 (EP) 2214609.6

(51) Int. Cl. A61K 31/553; A61K 31/22; A61K 31/366; A61K 31/405; A61K 31/426; A61K 31/439; A61K 31/47; A61K 31/517; A61K 31/7036; A61P 33/02

(52) U.S. Cl. CPC A61K 31/553; A61K 31/22; A61K 31/366; A61K 31/405; A61K 31/426; A61K 31/439; A61K 31/47; A61K 31/517; A61K 31/7036; A61P 33/02

(57) ABSTRACT

The present invention relates to compositions and methods for treating or preventing infections, in particular infections by intracellular parasites such as Cryptosporidium spp. The present invention also relates to compositions and methods for treating or preventing viral or bacterial infections, in particular intestinal infections such as those caused by rotavirus and Salmonella spp. Infections. The methods comprise administration of, and the compositions comprise, a farnesyl-diphosphate farnesyltransferase 1 (FDFT1) inhibitor, such as lapaquistat.

### FIELD OF THE INVENTION

The present invention relates to compositions and methods for treating clinical conditions arising from intestinal infections, in particular parasitic diseases such as those caused by Cryptosporidium spp. The present invention also relates to compositions and methods for treating clinical conditions arising from viral and bacterial infections, in particular intestinal diseases such as those caused by rotavirus and Salmonella spp. infections.

### BACKGROUND

The Cryptosporidium parasite is a leading cause of diarrheal illness in humans and livestock. In humans, immunocompromised patients and children bear a substantial burden of the disease. The only approved drug in humans is nitazoxanide, which has limited efficacy in malnourished and immunocompromised patients [1]. Complications from Cryptosporidium infection kills somewhere between 50-200K people per year (mostly children) and there is a strong correlation between early life cases and chronic malnutrition [2]. There are no vaccines available and there is a desperate need for more effective therapeutics to treat infection.

Cryptosporidium is a eukaryotic pathogen that specifically infects epithelial cells within the host intestine. As an intracellular parasite, Cryptosporidium is entirely dependent on its host cell for nutrients and shelter while it grows and replicates. Replication by the parasite is both asexual (mitosis) and sexual (meiosis) and the latter is required for production of new oocysts, which are the transmissible form of the parasite. In order to identify the host genes and pathways that are essential for supporting parasite growth and development, the present inventors employed a high throughput microscopy-based CRISPR screen across the entire human genome. From this screen we identified the host gene FDFT1, or farnesyl-diphosphate farnesyltransferase 1, as essential for the parasite to grow and develop. Further, we identified two inhibitors of FDFT1, TAK-475 or lapaquistat, and YM-53601 that are capable of blocking Cryptosporidium growth and development. Lapaquistat is described in detail in U.S. Pat. No. 6,613,761B1, which is incorporated herein by reference. YM-53601 is described in detail in document [3], which is incorporated herein by reference.

### SUMMARY OF THE INVENTION

The present invention provides a farnesyl-diphosphate farnesyltransferase 1 (FDFT1) inhibitor, or a pharmaceutically acceptable salt thereof for use in a method of treating or preventing an infection. In some embodiments, the infection is an infection by an intracellular pathogen which is incapable of endogenous cholesterol biosynthesis. In some embodiments, the infection is an infection by an intracellular pathogen which is incapable of endogenous squalene synthesis. In some embodiments, the infection is an infection by an intracellular pathogen which does not express an endogenous squalene synthase.

The present invention provides a farnesyl-diphosphate farnesyltransferase 1 (FDFT1) inhibitor, or a pharmaceutically acceptable salt thereof for use in a method of treating or preventing a parasitic infection.

The inventors have surprisingly found that effective inhibition of parasite growth can be achieved by targeting the cholesterol biosynthesis pathway of the host (as opposed to cholesterol biosynthesis in the parasite), specifically by inhibiting FDFT1 (squalene synthase) activity. Lapaquistat is a suitable FDFT1 inhibitor which can be used to treat a parasitic infection (e.g., a Cryptosporidium infection) in accordance with the methods of the invention, as demonstrated in, e.g., Example 3. Because of the unique mechanism of action, it is expected that FDFT1 inhibition would also be an effective treatment against other pathogens that rely on host cells for cholesterol biosynthesis, in particular other intracellular pathogens that rely on host cells for cholesterol biosynthesis.

In some embodiments, the parasitic infection is an infection by an intracellular parasite which is incapable of endogenous cholesterol biosynthesis. In some embodiments, the parasitic infection is an infection by an intracellular parasite which is incapable of endogenous squalene synthesis. In some embodiments, the parasitic infection is an infection by an intracellular parasite which does not express an endogenous squalene synthase. In some embodiments, the parasitic infection is an infection by an intracellular parasite which is an obligate intracellular parasite.

In some embodiments, the parasitic infection is an infection by an intracellular parasite which is in the order of Eucoccidiorida, preferably in the suborder of Eimeriorina. Preferably, the parasite is a Cryptosporidium spp.

The present invention also provides a farnesyl-diphosphate farnesyltransferase 1 (FDFT1) inhibitor, or a pharmaceutically acceptable salt thereof for use in a method of treating or preventing a Cryptosporidium infection.

The present invention also provides a farnesyl-diphosphate farnesyltransferase 1 (FDFT1) inhibitor, or a pharmaceutically acceptable salt thereof for use in a method of treating or preventing a viral infection. In some embodiments, the viral infection is an infection by an enveloped virus. As viruses are intracellular pathogens that are incapable of endogenous cholesterol biosynthesis, it is expected that FDFT1 inhibition would be effective in treating viral infections, particularly enveloped viruses that rely on host cell cholesterol biosynthesis.

The present invention also provides a farnesyl-diphosphate farnesyltransferase 1 (FDFT1) inhibitor, or a pharmaceutically acceptable salt thereof for use in a method of treating or preventing a rotavirus infection.

The present invention also provides a farnesyl-diphosphate farnesyltransferase 1 (FDFT1) inhibitor, or a pharmaceutically acceptable salt thereof for use in a method of treating or preventing a bacterial infection. In some embodiments, the bacterial infection is an infection by an intracellular bacterium which is incapable of endogenous cholesterol biosynthesis. In some embodiments, the bacterial infection is an infection by an intracellular bacterium which is incapable of endogenous squalene synthesis. In some embodiments, the bacterial infection is an infection by an intracellular bacterium which does not express an endogenous squalene synthase.

The present invention also provides a farnesyl-diphosphate farnesyltransferase 1 (FDFT1) inhibitor, or a pharmaceutically acceptable salt thereof for use in a method of treating or preventing a Salmonella infection.

In some embodiments, the FDFT1 inhibitor is lapaquistat, or a pharmaceutically acceptable salt thereof. In some embodiments, the FDFT1 inhibitor has the following structure:

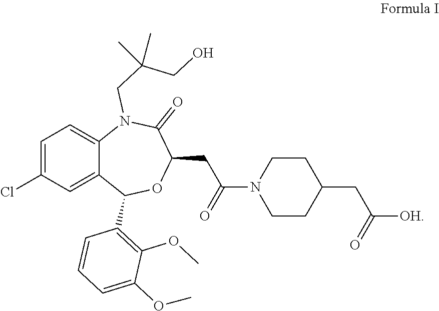 [SMILES: COc1cccc([C@H]2O[C@H](CC(=O)N3CCC(CC(=O)O)CC3)C(=O)N(CC(C)(C)CO)c3ccc(Cl)cc32)c1OC]

In some embodiments, the FDFT1 inhibitor is lapaquistat.

In some embodiments, the FDFT1 inhibitor is YM 53601, or a pharmaceutically acceptable salt thereof. In some embodiments, the FDFT1 inhibitor has the following structure:

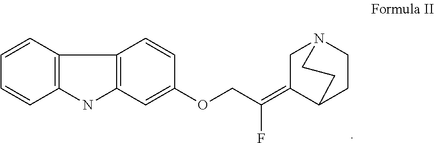 [SMILES: F/C(COc1ccc2c(c1)[nH]c1ccccc12)=C1/CN2CCC1CC2]

In some embodiments, the FDFT1 inhibitor is administered simultaneously, sequentially or separately with one or more other drugs used in the treatment or prophylaxis of the parasitic infection or Cryptosporidium infection. In some embodiments, the one or more other drugs is formulated in a combined preparation with the FDFT1 inhibitor or pharmaceutically acceptable salt thereof.

In some embodiments, the one or more other drugs comprises nitazoxanide, paromomycin or halofuginone. In some embodiments, the one or more other drugs comprises nitazoxanide.

The inventors surprisingly found that inhibition of parasite growth was synergistically improved when the FDFT1 inhibitor (e.g., lapaquistat) was combined with a statin (e.g., lovastatin or atorvastatin), as described in Example 5. Thus, in some embodiments, the FDFT1 inhibitor is administered simultaneously, sequentially or separately with one or more other drugs which inhibit cholesterol biosynthesis and/or which reduce the amount of cholesterol in the blood of the subject. In some embodiments, the one or more other drugs which inhibit cholesterol biosynthesis and/or which reduce the amount of cholesterol in the blood of the subject is a statin. In some embodiments, the statin is selected from one or more of lovastatin, atorvastatin, fluvastatin, pitavastatin, pravastatin, rosuvastatin, and simvastatin. In some embodiments, the statin is lovastatin or atorvastatin.

In some embodiments, the FDFT1 inhibitor is lapaquistat wherein the lapaquistat is administered simultaneously, sequentially or separately with lovastatin or atorvastatin.

The present invention also provides a statin for use in a method of treating or preventing an infection, wherein the statin is administered simultaneously, sequentially or separately with a FDFT1 inhibitor, or a pharmaceutically acceptable salt thereof.

The present invention provides the combination of an FDFT1 inhibitor, or a pharmaceutically acceptable salt thereof, and a drug which inhibits cholesterol biosynthesis and/or which reduces the amount of cholesterol in the blood of the subject. The combination may be for use in a method of treating or preventing an infection. In some embodiments, the infection is an infection by an intracellular pathogen which is incapable of endogenous cholesterol biosynthesis. In some embodiments, the infection is an infection by an intracellular pathogen which is incapable of endogenous squalene synthesis. In some embodiments, the infection is an infection by an intracellular pathogen which does not express an endogenous squalene synthase. The infection may be an intracellular parasitic, viral or bacterial invention. The infection may preferably be an intracellular parasitic infection. The infection may be a Cryptosporidium infection, a rotavirus infection, or a Salmonella infection. The infection may preferably by a Cryptosporidium infection. The FDFT1 inhibitor or pharmaceutically acceptable salt thereof may be lapaquistat or YM 53601. The drug which inhibits cholesterol biosynthesis and/or which reduces the amount of cholesterol in the blood of the subject may be a statin, for example a statin selected from the group consisting of lovastatin, atorvastatin, fluvastatin, pitavastatin, pravastatin, rosuvastatin, and simvastatin. In some preferred embodiments, the statin is lovastatin or atorvastatin.

In embodiments relating to a combination of an FDFT1 inhibitor, or a pharmaceutically acceptable salt thereof, and a drug which inhibits cholesterol biosynthesis and/or which reduces the amount of cholesterol in the blood of the subject, the two components may be for simultaneous, sequential or separate administration. The FDFT1 inhibitor or pharmaceutically acceptable salt thereof may be lapaquistat or YM 53601. The drug which inhibits cholesterol biosynthesis and/or which reduces the amount of cholesterol in the blood of the subject may be a statin, for example a statin selected from the group consisting of lovastatin, atorvastatin, fluvastatin, pitavastatin, pravastatin, rosuvastatin, and simvastatin. In some preferred embodiments, the statin is lovastatin or atorvastatin.

Other combinations are also contemplated. For example, in the case of parasitic infections, the FDFT1 inhibitor, or a pharmaceutically acceptable salt thereof, may be combined with one or more other drugs used in the treatment or prophylaxis of a parasitic infection, such as a Cryptosporidium infection. Such drugs may be nitazoxanide, paromomycin or halofuginone. In some embodiments, the additional drug is nitazoxanide, Triple combinations of an FDFT1 inhibitor, or a pharmaceutically acceptable salt thereof, and a drug which inhibits cholesterol biosynthesis and/or which reduces the amount of cholesterol in the blood of the subject (such as a statin), and one or more other drugs used in the treatment or prophylaxis of a Cryptosporidium infection (such as nitazoxanide, paromomycin or halofuginone) are also contemplated. Components of any combination may be for simultaneous, separate, or sequential administration.

There is also provided a composition comprising an FDFT1 inhibitor, or a pharmaceutically acceptable salt thereof, and a drug which inhibits cholesterol biosynthesis and/or which reduces the amount of cholesterol in the blood of the subject. The composition may be for use in a method of treating or preventing an infection. In some embodiments, the infection is an infection by an intracellular pathogen which is incapable of endogenous cholesterol biosynthesis. In some embodiments, the infection is an infection by an intracellular pathogen which is incapable of endogenous squalene synthesis. In some embodiments, the infection is an infection by an intracellular pathogen which does not express an endogenous squalene synthase. The infection may be an intracellular parasitic, viral or bacterial invention. The infection may preferably be an intracellular parasitic infection. The infection may be a Cryptosporidium infection, a rotavirus infection, or a Salmonella infection. The infection may preferably by a Cryptosporidium infection. The FDFT1 inhibitor or pharmaceutically acceptable salt thereof may be lapaquistat or YM 53601. The drug which inhibits cholesterol biosynthesis and/or which reduces the amount of cholesterol in the blood of the subject may be a statin, for example a statin selected from the group consisting of lovastatin, atorvastatin, fluvastatin, pitavastatin, pravastatin, rosuvastatin, and simvastatin. In some preferred embodiments, the statin is lovastatin or atorvastatin. The composition may further comprise one or more other drugs used in the treatment or prophylaxis of a parasitic infection, such as a Cryptosporidium infection. Such drugs may be nitazoxanide, paromomycin or halofuginone.

There is also provided a kit of parts comprising an FDFT1 inhibitor, or a pharmaceutically acceptable salt thereof, and a drug which inhibits cholesterol biosynthesis and/or which reduces the amount of cholesterol in the blood of the subject. The FDFT1 inhibitor, or a pharmaceutically acceptable salt thereof, and the drug which inhibits cholesterol biosynthesis and/or which reduces the amount of cholesterol in the blood of the subject may be disposed separately in separate containers. The kit of parts may further comprise instructions for use. For example, the instructions for use may be for treating or preventing an infection. In some embodiments, the infection is an infection by an intracellular pathogen which is incapable of endogenous cholesterol biosynthesis. In some embodiments, the infection is an infection by an intracellular pathogen which is incapable of endogenous squalene synthesis. In some embodiments, the infection is an infection by an intracellular pathogen which does not express an endogenous squalene synthase. The infection may be an intracellular parasitic, viral or bacterial invention. The infection may preferably be an intracellular parasitic infection. The infection may be a Cryptosporidium infection, a rotavirus infection, or a Salmonella infection. The infection may preferably by a Cryptosporidium infection. The FDFT1 inhibitor or pharmaceutically acceptable salt thereof may be lapaquistat or YM 53601. The drug which inhibits cholesterol biosynthesis and/or which reduces the amount of cholesterol in the blood of the subject may be a statin, for example a statin selected from the group consisting of lovastatin, atorvastatin, fluvastatin, pitavastatin, pravastatin, rosuvastatin, and simvastatin. In some preferred embodiments, the statin is lovastatin or atorvastatin. The kit may further comprise one or more other drugs used in the treatment or prophylaxis of a parasitic infection, such as a Cryptosporidium infection. Such drugs may be nitazoxanide, paromomycin or halofuginone.

In some embodiments, the FDFT1 inhibitor is administered to the patient orally, intravenously, intramuscularly, intrathecally, subcutaneously, sublingually, buccally, rectally, vaginally, ocularly, oticly, nasally, by inhalation, by nebulization, cutaneously, topically or transdermally. In some embodiments, the subject is a human. In some embodiments, the subject is a cow (cattle), a sheep, a goat, a cat, a dog, a pig (swine), a horse or a rabbit.

In some embodiments, the FDFT1 inhibitor is administered in a dose sufficient to achieve at least 50%, at least 60%, at least 70%, at least 80%, at least 90%, at least 95% or at least 99% inhibition of FDFT1 activity. In some embodiments, the FDFT1 inhibitor is administered in a dose sufficient to achieve at least 50%, at least 60%, at least 70%, at least 80%, at least 90%, at least 95% or at least 99% inhibition of squalene production in a target cell. In some embodiments, the FDFT1 inhibitor prevents sexual development of the parasite. In some embodiments, the FDFT1 inhibitor reduces the reproduction of the parasite.

In some embodiments, the method comprises administering to patient the FDFT1 inhibitor or pharmaceutically acceptable salt thereof at a dose of 0.0001 mg/kg to 100 mg/kg. In some embodiments, the method comprises administering a single daily dose of 25 mg to 100 mg of the FDFT1 inhibitor or pharmaceutically acceptable salt thereof to the patient.

The present invention also provides for use of a therapeutically or prophylactically effective amount of a farnesyl-diphosphate farnesyltransferase 1 (FDFT1) inhibitor, or a pharmaceutically acceptable salt thereof in a method of treating or preventing a parasitic infection.

The present invention also provides for use of a therapeutically or prophylactically effective amount of a farnesyl-diphosphate farnesyltransferase 1 (FDFT1) inhibitor, or a pharmaceutically acceptable salt thereof in a method of treating or preventing a Cryptosporidium infection.

Lapaquistat already has substantial human safety data (>3 large clinical trials). There is strong potential for repurposing lapaquistat for treatment of parasitic diseases, in particular Cryptosporidium treatment. Cryptosporidium treatment would not require chronic dosing and because lapaquistat has low bioavailability we believe the drug will concentrate in the intestine—at the site of infection—and increase potency allowing for a low dose to be used.

Infections treated in the present invention may be intracellular infections, i.e. infections caused by intracellular pathogens. In such infections, the pathogen enters and replicates within the host's cells, typically using the host cell's machinery to reproduce and spread. This is in contrast to extracellular infections, where pathogens remain outside the host cells.

### BRIEF DESCRIPTION OF THE DRAWINGS

FIG. 1—FDFT1 inhibition as a treatment for Cryptosporidium. A) Schema of microscopy-based whole genome CRISPR-Cas9 screen to identify host factors essential for growth and development of the Cryptosporidium parasite. B) Median z-scores from 3 biological replicates. Note that FDFT1 is identified as highly significant in reducing total parasite numbers (left—total parasites/total host nuclei) and the highest z-score in reducing parasite development (right—total female parasites/total parasites). C) Lapaquistat treatment of infected intestinal epithelial cells show efficacy against parasite growth and parasite development, measured by % female parasites in total parasites identified.

FIG. 2—FDFT1 inhibition with YM-53601 as a treatment for Cryptosporidium. YM-53601 treatment of infected intestinal epithelial cells show efficacy against parasite growth and parasite development, measured by % female parasites in total parasites identified.

FIG. 3—Synergistic combination of FDFT1 inhibition and statins. Intestinal epithelial cells (HCT-8) were infected with Cryptosporidium parasites and treated with increasing concentrations of Lapaquistat acetate or Lovastatin, either alone or in combination. The potency of Lapaquistat, with respect to its ability to limit Cryptosporidium growth, was synergistically increased when in combination with Lovastatin.

### DETAILED DESCRIPTION OF THE INVENTION

### Definitions

Below are provided certain definitions of terms, technical means, and embodiments used herein.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%), 6%), 5%, 4%, 3%, 2%, 1%), or less in either direction (greater than or less than) of the stated reference value unless otherwise stated or otherwise evident from the context (except where such number would exceed 100% of a possible value).

As used herein, the term “amelioration” means the prevention, reduction or palliation of a state, or improvement of the state of a subject or in disease biomarkers of severity or outcome. Amelioration includes, but does not require, complete recovery or complete prevention of a disease condition.

The term “comparable”, as used herein, refers to a system, set of conditions, effects, or results that is/are sufficiently similar to a test system, set of conditions, effects, or results, to permit scientifically legitimate comparison. Those of ordinary skill in the art will appreciate and understand which systems, sets of conditions, effects, or results are sufficiently similar to be “comparable” to any particular test system, set of conditions, effects, or results as described herein.

The term “correlates”, as used herein, has its ordinary meaning of “showing a correlation with”. Those of ordinary skill in the art will appreciate that two features, items or values show a correlation with one an-other if they show a tendency to appear and/or to vary, together. In some embodiments, a correlation is statistically significant when its p-value is less than 0.05; in some embodiments, a correlation is statistically significant when its p-value is less than 0.01. In some embodiments, correlation is assessed by regression analysis. In some embodiments, a correlation is a correlation coefficient.

As used herein, the terms “improve,” “increase” or “reduce,” or grammatical equivalents, indicate values that are relative to a reference (e.g., baseline) measurement, such as a measurement taken under comparable conditions (e.g., in the same individual prior to initiation of treatment described herein, or a measurement in a control individual (or multiple control individuals) in the absence of treatment) described herein.

As used herein, a “polypeptide”, generally speaking, is a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides sometimes include “non-natural” amino acids or other entities that nonetheless are capable of integrating into a polypeptide chain, optionally.

As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids.

As used herein, the term “subject”, “individual”, or “patient” refers to any organism upon which embodiments of the invention may be used or administered, e.g., for experimental, diagnostic, prophylactic, and/or therapeutic purposes. These terms include mammals, such as humans and both domestic animals such as laboratory animals and household pets (e.g., cats, dogs, swine, cattle, sheep, goats, horses, rabbits), and non-domestic animals such as wildlife and the like. In an exemplary aspect of the present invention, to identify subject patients for treatment according to the methods of the invention, accepted screening methods are employed to determine risk factors associated with a targeted or suspected disease or condition or to determine the status of an existing disease or condition in a subject. These screening methods include, for example, conventional diagnostic techniques associated with the targeted or suspected disease or condition. These and other routine methods allow the clinician to select patients in need of therapy using the methods and formulations of the present invention. In a preferred embodiment of the invention the subject is a human. In some embodiments of the invention the subject is a domestic animal such as a cat, a dog, a pig (swine), a cow (cattle), a sheep, a goat, a horse or a rabbit. In some embodiments of the invention the subject is a cow (cattle). In some embodiments of the invention the subject is a sheep. In some embodiments of the invention the subject is a goat. In some embodiments of the invention the subject is a cat. In some embodiments of the invention the subject is a dog. In some embodiments of the invention the subject is a pig (swine).

As used herein, the term “therapeutic regimen” refers to any method used to partially or completely alleviate, ameliorate, relieve, inhibit, prevent, delay onset of, reduce severity of and/or reduce incidence of one or more symptoms or features of a particular disease, disorder, and/or condition. It may include administration of one or more doses, optionally spaced apart by regular or varied time intervals. In some embodiments, a therapeutic regimen is one whose performance is designed to achieve and/or is correlated with achievement of (e.g., across a relevant population of cells, tissues, or organisms) a particular effect, e.g., reduction or elimination of a detrimental condition or disease. In some embodiments, treatment includes administration of one or more therapeutic agents either simultaneously, sequentially or at different times, for the same or different amounts of time. In some embodiments, a “treatment regimen” includes genetic methods such as gene therapy, gene ablation or other methods known to induce or reduce expression (e.g. transcription, processing, and/or translation of a particular gene product, such as a primary transcript or mRNA).

As used herein, the term “therapeutically effective amount” refers to an amount of a therapeutic agent which confers a therapeutic effect on the treated subject, at a reasonable benefit/risk ratio applicable to any medical treatment. Such a therapeutic effect may be objective (i.e., measurable by some test or marker) or subjective (i.e., subject gives an indication of or feels an effect). In some embodiments, “therapeutically effective amount” refers to an amount of a therapeutic agent or composition effective to treat, ameliorate, or prevent (e.g., delay onset of or reduce risk of) a relevant disease or condition, and/or to exhibit a detectable therapeutic or preventative effect, such as by ameliorating symptoms associated with the disease, preventing or delaying onset of the disease, and/or also lessening severity or frequency of symptoms of the disease. A therapeutically effective amount is commonly administered in a dosing regimen that may comprise multiple unit doses. For any particular therapeutic agent, a therapeutically effective amount (and/or an appropriate unit dose within an effective dosing regimen) may vary, for example, depending on route of administration, or on combination with other therapeutic agents. Alternatively or additionally, a specific therapeutically effective amount (and/or unit dose) for any particular patient may depend upon a variety of factors including the activity of the specific therapeutic agent employed; the specific composition employed; the age, body weight, general health, sex and diet of the patient; the time of administration, route of administration, and/or rate of excretion or metabolism of the specific therapeutic agent employed; the duration of the treatment; and like factors as is well known in the medical arts. The term “prophylactically effective amount” used herein refers to the amount of agent needed to prevent the parasitic or infectious disease or disorder as defined elsewhere herein.

Preferably, the dose of the agent is sufficient to inhibit the FDFT1 activity as much possible in the subject. The dose of the agent supplied may be e.g. about 1, 1.5, 2, 2.5 times, 3 times or 4 times the molar dose needed to inhibit FDFT1 activity in the subject. Preferably, the dose is from 0.0001 mg/kg (mass of drug compared to mass of patient) to 100 mg/kg, preferably 0.001 mg/kg to 50 mg/kg, preferably 0.001 mg/kg to 20 mg/kg, preferably 0.001 mg/kg to 10 mg/kg, preferably 0.01 mg/kg to 5 mg/kg, preferably 0.01 mg/kg to 2 mg/kg, preferably 0.1 mg/kg to 1.5 mg/kg; alternatively 0.2 mg/kg to 1.2 mg/kg; The therapeutically or prophylactically effective amount can additionally be defined in terms of the inhibition of FDFT1 activity, for example, an amount that means that FDFT1 activity is reduced by at least 10, 20, 30, 40, 50, 60, 70, 80, 90, 91, 92, 93, 94, 95, 96, 97, 98, 99, 100%, compared to FDFT1 activity in the absence of treatment. Dose and frequency may be adjusted in order to maintain FDFT1 activity at the desired level, which may be, for example 10% or less, for example 9, 8, 7, 6, 5, 4, 3, 2, 1% or less compared to FDFT1 activity in the absence of treatment. Where a dose is given, this relates to a dose of the agent which is a FDFT1 inhibitor or pharmaceutically acceptable salt thereof.

FDFT1 activity can be measured by standard assays known in the art, e.g. by measuring squalene production as a proxy measure of FDFT1 activity. Suitable methods of measuring squalene synthesis are known in the art [3,4,5], for example by measuring the radioactive product of liver microsomes incubated with tritiated farnesyl pyrophosphate (FPP).

The frequency with which the dose needs to be administered will depend on the half-life of the agent involved. The FDFT1 inhibitor or pharmaceutically acceptable salt thereof, may be administered as a continuous infusion, in bolus doses or on a daily basis, twice daily basis, or every two, three, four days, five, six, seven, 10, 15 or 20 days or more. In a preferred embodiment the FDFT1 inhibitor or pharmaceutically acceptable salt thereof, is administered in a bolus dose on a daily basis.

The FDFT1 inhibitor or pharmaceutically acceptable salt thereof, may be administered for a suitable period of time as determined by a clinician. The FDFT1 inhibitor or pharmaceutically acceptable salt thereof, may be administered for up to 30 days, up to 21 days, up to 14 days or up to 7 days, for example for up to 1, 2, 3, 4, 5, 6 or 7 days. The FDFT1 inhibitor or pharmaceutically acceptable salt thereof, may be administered daily for up to 30 days, up to 21 days, up to 14 days or up to 7 days, for example for up to 1, 2, 3, 4, 5, 6 or 7 days.

Single or multiple doses may be administered. For example, at least 2, 3, 4, 5, 6, 7, or 8 doses may be administered. Single doses are one embodiment. The exact dosage and the frequency of doses may also be dependent on the patient's status at the time of administration. Factors that may be taken into consideration when determining dosage include the need for treatment or prophylaxis, the severity of the disease state in the patient, the general health of the patient, the age, weight, gender, diet, time and frequency of administration, drug combinations, reaction sensitivities and the patient's tolerance or response to therapy. The precise amount can be determined by routine experimentation, but may ultimately lie with the judgement of the clinician.

As used herein, the term “treatment” (also “treat” or “treating”) refers to any administration of a therapeutic agent according to a therapeutic regimen that achieves a desired effect in that it partially or completely alleviates, ameliorates, relieves, inhibits, delays onset of, reduces severity of and/or reduces incidence of one or more symptoms or features of a particular disease, disorder, and/or condition. In some embodiments, administration of the therapeutic agent according to the therapeutic regimen is correlated with achievement of the desired effect. Such treatment may be of a subject who does not exhibit signs of the relevant disease, disorder and/or condition and/or of a subject who exhibits only early signs of the dis-ease, disorder, and/or condition. Alternatively, or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

As used herein “prevent” refers to delaying or forestalling the onset, development or progression of a disease, disorder, or condition for a period of time from minutes to indefinitely. “Prevent” also means reducing the risk of developing a disease, disorder, or condition.

As used herein, “modulation” means a change of amount or quality of a molecule, function, or activity when compared to the amount or quality of a molecule, function, or activity prior to modulation. For example, modulation includes the change, either an increase (stimulation or induction) or a decrease (inhibition or reduction) in gene expression.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The form of the pharmaceutical compositions, the route of administration, the dosage and the regimen naturally depend upon the condition to be treated, the severity of the illness, the age, weight, and sex of the patient, etc. In some embodiments, a pharmaceutically acceptable carrier or diluent is sterile saline. In some embodiments, such sterile saline is pharmaceutical grade saline.

The term “target cell” refers to a particular cell type of interest in which the functional consequence of FDFT1 inhibition may be desirable. In some embodiments, the target cell is an intestinal cell. In some embodiments, the target cell is an intestinal epithelial cell.

The term “comprising” is used herein to mean including the method steps or elements identified, but that such steps or elements do not comprise an exclusive list and as such there may be present additional steps or elements.

Further, to the extent that the term “includes” is used in either the detailed description or the claims, such term is intended to be inclusive in a manner similar to the term “comprising” as “comprising” is interpreted when employed as a transitional word in a claim.

In some embodiments, the FDFT1 inhibitor comprises a small molecule, such as lapaquistat as described herein. In some embodiments, the FDFT1 inhibitor comprises a small molecule, such as YM-53601 as described herein. In some embodiments, the FDFT1 inhibitor is an inhibitory nucleic acid molecule (e.g. siRNA). In some embodiments, the FDFT1 inhibitor comprises an antibody or antigen binding fragment thereof.

### Parasitic Diseases

The FDFT1 inhibitors or pharmaceutically acceptable salts thereof of the present invention may be used in the treatment of parasitic infections. In particular embodiments of the invention, the present invention relates to treatment of intestinal parasitic infections. Such parasitic infections may include:
(a) infections by single-celled parasites, for example, Cryptosporidiosis caused by Cryptosporidium parvum, Cryptosporidium hominis, Cryptosporidium canis, Cryptosporidium felis, Cryptosporidium meleagridis, and Cryptosporidium bovis.

In particular, the present invention relates to the treatment of Cryptosporidiosis caused by Cryptosporidium parvum, Cryptosporidium hominis, Cryptosporidium canis, Cryptosporidium felis, Cryptosporidium meleagridis, and Cryptosporidium bovis. In particular, the present invention relates to the treatment of Cryptosporidiosis caused by Cryptosporidium parvum.

Other parasitic infections that may be treated include those caused by other intracellular parasites which are incapable of endogenous cholesterol biosynthesis. In particular, parasites that do not express an endogenous squalene synthase.

### Viral Diseases

The FDFT1 inhibitors or pharmaceutically acceptable salts thereof of the present invention may be used in the treatment of viral infections. In particular embodiments of the invention, the present invention relates to treatment of intestinal viral infections. Such viral infections may include:
(a) Rotaviruses, Norwalk virus, Noroviruses (Norwalk-like viruses), Adenoviruses, Astroviruses, other Caliciviruses and Parvoviruses.

In a particular embodiment of the invention The FDFT1 inhibitors or pharmaceutically acceptable salts thereof of the present invention may be used in the treatment of a rotavirus infection.

Viral gastroenteritis is a very common cause of diarrhoea. Food-borne and water-borne epidemics of viral gastroenteritis are common. The US Centers for Disease Control and Prevention (CDC) estimate that viruses cause 9.2 million (out of a total of 13.8 million from all causes) cases of food-related illness each year.

Several different viruses can cause this disease including Rotavirus, Adenovirus, Astrovirus, Calicivirus (Noroviruses and Norwalk virus). Two major causes of human viral gastroenteritis (Rotavirus and Norwalk virus) are carried in the intestine of most domestic and many wild animals. Fecal-oral spread from animal-to-person or person-to-person can cause diarrhoea. Rotavirus disease (winter diarrhoea) is most common among infants and young children while Norwalk virus disease (summer diarrhoea) affects older children and adults.

Rotavirus is in the Reoviridae family, i.e., it is a naked double-stranded RNA virus with a double icosahedral capsid.

The Norwalk virus is in the Caliciviridae family, i.e., it is a naked single stranded RNA-containing virus with a single icosahedral capsid. Noroviruses (i.e., Norwalk-like viruses or NLV) are members of the family Caliciviridae and are well-recognized etiologic agents of nonbacterial acute gastroenteritis. The virus is transmitted by hands contaminated through the fecal-oral route, directly from person to person, through contaminated food or water, or by contact with contaminated surfaces or fomites. Adenoviruses serotypes 40 and 41 are most commonly associated with diarrhoea in infants.

The viruses invade and destroy mature epithelial cells in the middle and upper villus, causing a decreased absorption of sodium and water from the bowel lumen.

Symptoms include low-grade fever, abdominal pain, watery diarrhoea, nausea and vomiting. Rotaviruses usually cause vomiting diarrhoea and fever in babies less than 2 years of age. The elderly can also have problems with this virus. The incubation period being is 2-4 days. The diarrhoea can last for extended periods of time resulting in dehydration.

Adenoviruses cause symptoms similar to rotavirus infections except that the infants tend to be older. Complications can include intussusception. Norovirus symptoms last 12-60 hours and are characterized by sudden onset of low-grade fever, nausea, vomiting, and watery diarrhoea; the incubation period is 12-48 hours. These viruses can infect children and adults.

In one embodiment of the invention, the FDFT1 inhibitors or pharmaceutically acceptable salt thereof as described herein treat or ameliorate one or more symptoms of intestinal viral infections as set out above.

### Bacterial Diseases

The FDFT1 inhibitors or pharmaceutically acceptable salts thereof of the present invention may be used in the treatment of bacterial infections. In particular embodiments of the invention, the present invention relates to treatment of intestinal bacterial infections. Such bacterial infections may include:
(a) Salmonella, such as diseases caused by Salmonella enterica or Salmonella bongori.

In a particular embodiment of the invention The FDFT1 inhibitors or pharmaceutically acceptable salts thereof of the present invention may be used in the treatment of a bacterial infection. In a particular embodiment of the invention The FDFT1 inhibitors or pharmaceutically acceptable salts thereof of the present invention may be used in the treatment of a Salmonella infection.

Salmonella species are facultative intracellular pathogens. Salmonella can invade different cell types, including epithelial cells, M cells, macrophages, and dendritic cells.

Symptoms of Salmonella include back pain or spondylosis. It can manifest as five clinical patterns: gastrointestinal tract infection, enteric fever, bacteremia, local infection, and the chronic reservoir state. The initial symptoms are nonspecific fever, weakness, and myalgia among others. In the bacteremia state, it can spread to any parts of the body and this induces localized infection or it forms abscesses. Some forms of localized Salmonella infections are arthritis, urinary tract infection, infection of the central nervous system, bone infection, soft tissue infection. Infection may remain as the latent form for a long time, and when the function of reticular endothelial cells is deteriorated, it may become activated and consequently, it may secondarily induce spreading infection in the bone several months or several years after acute salmonellosis.

In one embodiment of the invention, the FDFT1 inhibitors or pharmaceutically acceptable salt thereof as described herein treat or ameliorate one or more symptoms of intestinal bacterial infections as set out above. In one embodiment of the invention, the FDFT1 inhibitors or pharmaceutically acceptable salt thereof as described herein treat or ameliorate one or more symptoms of Salmonella infections as set out above.

### Cryptosporidiosis

Common signs and symptoms of intestinal cryptosporidiosis include:
Moderate to severe watery diarrhoea, sometimes contains mucus and rarely contains blood or leukocytes (in very severe cases, diarrhoea may be profuse and cholera-like with malabsorption and hypovolemia)
Low-grade fever
Crampy abdominal pain
Dehydration
Weight loss
Fatigue
Nausea and vomiting
Epigastric or right upper quadrant tenderness

Other symptoms may include:
Reactive arthritis (may affect the hands, knees, ankles, and feet)
Jaundice
Ascites

Symptoms of upper respiratory cryptosporidiosis include:
Inflammation of the nasal mucosa, sinuses, larynx, or trachea
Nasal discharge
Voice change (e.g., hoarseness)

Symptoms of lower respiratory cryptosporidiosis include:
Cough
Shortness of breath
Fever
Hypoxemia

In one embodiment of the invention, the FDFT1 inhibitors or pharmaceutically acceptable salt thereof as described herein treat or ameliorate one or more symptoms of cryptosporidiosis set out above.

### Pharmaceutical Compositions of the Agent

As used herein “pharmaceutical composition” means a mixture of substances suitable for administering to an individual. A pharmaceutical composition may comprise one or more pharmaceutically active ingredients in combination with one more or pharmaceutically acceptable excipients or diluents. For example, a pharmaceutical composition may comprise one or more active pharmaceutical agents and a sterile aqueous solution.

As used herein “pharmaceutically acceptable salts” means physiologically and pharmaceutically acceptable salts of FDFT1 inhibitors, i.e., salts that retain the desired biological activity of the FDFT1 inhibitor and do not impart undesired toxicological effects thereto. Such salts include acetate, aspartate, benzoate, besylate, bromide/hydrobromide, bicarbonate/carbonate, bisulfate/sulfate, camphorsulfonate, chloride/hydrochloride, chlortheophyllonate, citrate, ethandisulfonate, fumarate, gluceptate, gluconate, glucuronate, oleate, oxalate, palmitate, pamoate, phosphate/hydrogen phosphate/dihydrogen phosphate, propionate, stearate, succinate, sulfosalicylate, tartrate, tosylate, trifluoroacetate salt or the like. Inorganic acids from which salts can be derived include, for example, hydrochloric acid, hydrobromic acid, sulfuric acid, nitric acid, phosphoric acid, and the like. In one embodiment, the salt is an acetate salt.

Compounds of the present invention may include their isomers, salts and solvates. Certain compounds of the invention may exist in one or more particular geometric, tautomeric, conformational, or anomeric forms, including but not limited to, cis- and transforms, E- and Z-forms, C-, t-, and r-forms, endo- and exo-forms, keto-, enol-, and enolate-forms, syn- and anti-forms, synclinal- and anticlinal-forms, [alpha]- and [beta]-forms, axial and equatorial forms, boat-, chair-, twist-, envelope-, and halfchair-forms, and combinations thereof, hereinafter collectively referred to as “isomers” (or “isomeric forms”).

The pharmaceutical composition, made in accordance with the present invention, can be prepared and administered in a wide variety of dosage forms. For example, these pharmaceutical compositions can be made in inert, pharmaceutically acceptable carriers which are either solid or liquid. Solid form preparations include powders, tablets, dispersible granules, capsules, cachets, and suppositories. Other solid and liquid form preparations could be made in accordance with known methods of the art and administered by the oral route in an appropriate formulation, or by a parenteral route such as intravenous, intramuscular, or subcutaneous injection as a liquid formulation.

The term “solvate” refers to a molecular complex of the compound with one or more solvent molecules. Such solvent molecules are those commonly used in the pharmaceutical art, which are known to be innocuous to the compound, e.g., water, ethanol, and the like.

Other aspects of the present invention also relate to a medicinal product or a diagnostic aid comprising a composition according to the invention or a FDFT1 inhibitor according to the invention and, where appropriate, suitable excipients and additives, such as, for example, a physiological saline solution, stabilizers or proteinase inhibitors.

Suitable pharmaceutically acceptable excipients would be known by the person skilled in the art, for example: fats, water, physiological saline, alcohol (e.g. ethanol), glycerol, polyols, aqueous glucose solution, extending agent, disintegrating agent, binder, lubricant, wetting agent, stabilizer, emulsifier, dispersant, preservative, sweetener, colorant, seasoning agent or aromatizer, concentrating agent, diluent, buffer substance, solvent or solubilizing agent, chemical for achieving storage effect, salt for modifying osmotic pressure, coating agent or antioxidant, saccharides such as lactose or glucose; starch of corn, wheat or rice; fatty acids such as stearic acid; inorganic salts such as magnesium metasilicate aluminate or anhydrous calcium phosphate; synthetic polymers such as polyvinylpyrrolidone or polyalkylene glycol; alcohols such as stearyl alcohol or benzyl alcohol; synthetic cellulose derivatives such as methylcellulose, carboxymethylcellulose, ethylcellulose or hydroxypropylmethylcellulose; and other conventionally used additives such as gelatin, talc, plant oil and gum arabic.

The pharmaceutical compositions may also contain preserving agents, solublizing agents, stabilizing agents, wetting agents, emulsifiers, sweeteners, colorants, odorants, buffers, coating agents and/or antioxidants.

### Combination Therapies

In some embodiments, the invention features a composition (e.g., one or more compositions, formulations or dosage formulations) or a pharmaceutical combination, comprising a FDFT1 inhibitor according to the invention and one or more further therapeutic agents. In some embodiments, the invention features a composition (e.g., one or more compositions, formulations or dosage formulations) or a pharmaceutical combination, comprising a therapeutic agent according to the invention and one or more further therapeutic agents.

In some embodiments, the one or more further therapeutic agents include a compound (i.e., drug) which inhibits cholesterol biosynthesis and/or which reduces the amount of cholesterol in the blood of the subject. For example, the one or more further therapeutic agents may include a statin, such as lovastatin or atorvastatin.

In some embodiments, the FDFT1 inhibitor is lapaquistat and is administered in combination with a statin, preferably lovastatin or atorvastatin.

In some embodiments, the composition comprises a pharmaceutically acceptable carrier or diluent. In some embodiments, the FDFT1 inhibitor and the one or more further therapeutic agents can be present in a single composition or as two or more different compositions. The FDFT1 inhibitor and the one or more further therapeutic agents can be administered via the same administration route or via different administration routes. The FDFT1 inhibitor and the one or more further therapeutic agents can be administered simultaneously or sequentially. In some embodiments, the pharmaceutical combination comprises the FDFT1 inhibitor and the one or more further therapeutic agents separately or together.

As used herein, the terms “combined treatment”, “combined therapy” or “therapy combination” refer to a treatment that uses more than one medication. The combined therapy may be dual therapy or bi-therapy.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

### Kits

The compounds and compositions of the invention can be packaged in a kit, optionally with one or more other pharmaceutical agents. Non-limiting examples of the kits include those that contain, e.g., two or more pills, a pill and a powder, a suppository and a liquid in a vial, or two topical creams. The kits can include optional components that aid in the administration of the unit dose to subjects, such as vials for reconstituting powder forms, syringes for injection, customized IV delivery systems, or inhalers. Additionally, the unit dose kits can contain instructions for preparation and administration of the compositions. The kits can be manufactured as a single use unit dose for one subject, multiple uses for a particular subject (at a constant dose or in which the individual compounds may vary in potency as therapy progresses); or the kits can contain multiple doses suitable for administration to multiple subjects (“bulk packaging”). The kit components can be assembled in cartons, blister packs, bottles, and tubes. Components of the kits may be disposed separately from each other, for example in separate containers. The kits may comprise instructions for use.

### Routes of Administration

The FDFT1 inhibitor or pharmaceutical composition can be administered by different routes including orally, parenterally, sublingually, intradermally, transdermally, rectally, transmucosally, topically, via inhalation, via buccal administration, intrapleurally, intravenously, intraarterially, intraperitoneally, subcutaneously, intramuscularly, intranasally, intrathecally, and/or intraarticularly, or combinations thereof. In some embodiments the agent or pharmaceutical composition is administered orally. In some embodiments the agent or pharmaceutical composition is administered intravenously.

The present invention is further illustrated in the following Examples. It should be understood that these Examples, while indicating embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### EXAMPLE, NON-LIMITING, COMBINATIONS

The present invention also extends to at least the following.

### FDFT1 Inhibitors and Statins

In the invention, the FDFT1 inhibitors may be used in combination with drugs that inhibit cholesterol biosynthesis and/or which reduce the amount of cholesterol in the blood of the subject (e.g. statins).

The present invention provides an FDFT1 inhibitor for use in the treatment of an infection by an intracellular pathogen that does not express an endogenous squalene synthase. The FDFT1 inhibitor may be combined with a statin for simultaneous, separate or sequential administration. The invention extends to corresponding methods of treatment, pharmaceutical compositions, combinations, and kits.

The present invention provides lapaquistat or YM53601 for use in the treatment of an infection by an intracellular pathogen that does not express an endogenous squalene synthase. The lapaquistat or YM53601 may be combined with a statin for simultaneous, separate or sequential administration. The invention extends to corresponding methods of treatment, pharmaceutical compositions, combinations, and kits.

The present invention provides lapaquistat or YM53601 for use in the treatment of a parasitic infection by an intracellular parasite that does not express an endogenous squalene synthase. The lapaquistat or YM53601 may be combined with a statin for simultaneous, separate or sequential administration. The invention extends to corresponding methods of treatment, pharmaceutical compositions, combinations, and kits.

The present invention provides lapaquistat or YM53601 for use in the treatment of a Cryptosporidium infection. The lapaquistat or YM53601 may be combined with a statin for simultaneous, separate or sequential administration. The invention extends to corresponding methods of treatment, pharmaceutical compositions, combinations, and kits.

The present invention provides lapaquistat or YM53601 for use in the treatment of a rotavirus infection. The lapaquistat or YM53601 may be combined with a statin for simultaneous, separate or sequential administration. The invention extends to corresponding methods of treatment, pharmaceutical compositions, combinations, and kits.

The present invention provides lapaquistat or YM53601 for use in the treatment of a Salmonella infection. The lapaquistat or YM53601 may be combined with a statin for simultaneous, separate or sequential administration. The invention extends to corresponding methods of treatment, pharmaceutical compositions, combinations, and kits.

The present invention provides lapaquistat for use in the treatment of an infection by an intracellular pathogen that does not express an endogenous squalene synthase. The lapaquistat may be combined with a statin for simultaneous, separate or sequential administration. The invention extends to corresponding methods of treatment, pharmaceutical compositions, combinations, and kits.

The present invention provides lapaquistat for use in the treatment of an intracellular parasitic infection by a parasite that does not express an endogenous squalene synthase. The lapaquistat may be combined with a statin for simultaneous, separate or sequential administration. The invention extends to corresponding methods of treatment, pharmaceutical compositions, combinations, and kits.

The present invention provides lapaquistat for use in the treatment of a Cryptosporidium infection. The lapaquistat may be combined with a statin for simultaneous, separate or sequential administration. The invention extends to corresponding methods of treatment, pharmaceutical compositions, combinations, and kits.

The present invention provides lapaquistat for use in the treatment of a rotavirus infection. The lapaquistat may be combined with a statin for simultaneous, separate or sequential administration. The invention extends to corresponding methods of treatment, pharmaceutical compositions, combinations, and kits.

The present invention provides lapaquistat for use in the treatment of a Salmonella infection. The lapaquistat may be combined with a statin for simultaneous, separate or sequential administration. The invention extends to corresponding methods of treatment, pharmaceutical compositions, combinations, and kits.

The present invention provides YM53601 for use in the treatment of an infection by an intracellular pathogen that does not express an endogenous squalene synthase. The YM53601 may be combined with a statin for simultaneous, separate or sequential administration. The invention extends to corresponding methods of treatment, pharmaceutical compositions, combinations, and kits.

The present invention provides YM53601 for use in the treatment of a parasitic infection by an intracellular parasite that does not express an endogenous squalene synthase. The YM53601 may be combined with a statin for simultaneous, separate or sequential administration. The invention extends to corresponding methods of treatment, pharmaceutical compositions, combinations, and kits.

The present invention provides YM53601 for use in the treatment of a Cryptosporidium infection. The YM53601 may be combined with a statin for simultaneous, separate or sequential administration. The invention extends to corresponding methods of treatment, pharmaceutical compositions, combinations, and kits.

The present invention provides YM53601 for use in the treatment of a rotavirus infection. The YM53601 may be combined with a statin for simultaneous, separate or sequential administration. The invention extends to corresponding methods of treatment, pharmaceutical compositions, combinations, and kits.

The present invention provides YM53601 for use in the treatment of a Salmonella infection. The YM53601 may be combined with a statin for simultaneous, separate or sequential administration. The invention extends to corresponding methods of treatment, pharmaceutical compositions, combinations, and kits.

The present invention provides the combination of:
a. an FDFT1 inhibitor; and
b. a statin;
   for use in the treatment of an infection by an intracellular pathogen that does not express an endogenous squalene synthase. The components of the combination may be for separate, simultaneous or sequential administration. The invention extends to corresponding uses, methods of treatment, pharmaceutical compositions, and kits.

The present invention provides the combination of:
a. lapaquistat or YM53601; and
b. a statin;
   for use in the treatment of an infection by an intracellular pathogen that does not express an endogenous squalene synthase. The components of the combination may be for separate, simultaneous or sequential administration. The invention extends to corresponding uses, methods of treatment, pharmaceutical compositions, and kits.

The present invention provides the combination of:
a. lapaquistat or YM53601; and
b. a statin;
   for use in the treatment of an intracellular parasitic infection by a parasite that does not express an endogenous squalene synthase. The components of the combination may be for separate, simultaneous or sequential administration. The invention extends to corresponding uses, methods of treatment, pharmaceutical compositions, and kits.

The present invention provides the combination of:
a. lapaquistat or YM53601; and
b. a statin;
   for use in the treatment of a Cryptosporidium infection. The components of the combination may be for separate, simultaneous or sequential administration. The invention extends to corresponding uses, methods of treatment, pharmaceutical compositions, and kits.

The present invention provides the combination of:
a. lapaquistat or YM53601; and
b. a statin;
   for use in the treatment of a rotavirus infection. The components of the combination may be for separate, simultaneous or sequential administration. The invention extends to corresponding uses, methods of treatment, pharmaceutical compositions, and kits.

The present invention provides the combination of:
a. lapaquistat or YM53601; and
b. a statin;
   for use in the treatment of a Salmonella infection. The components of the combination may be for separate, simultaneous or sequential administration. The invention extends to corresponding uses, methods of treatment, pharmaceutical compositions, and kits.

The present invention provides the combination of:
a. lapaquistat; and
b. a statin;
   for use in the treatment of a Cryptosporidium infection. The components of the combination may be for separate, simultaneous or sequential administration. The invention extends to corresponding uses, methods of treatment, pharmaceutical compositions, and kits.

The present invention provides the combination of:
a. lapaquistat; and
b. a statin;
   for use in the treatment of a rotavirus infection. The components of the combination may be for separate, simultaneous or sequential administration. The invention extends to corresponding uses, methods of treatment, pharmaceutical compositions, and kits.

The present invention provides the combination of:
a. lapaquistat; and
b. a statin;
   for use in the treatment of a Salmonella infection. The components of the combination may be for separate, simultaneous or sequential administration. The invention extends to corresponding uses, methods of treatment, pharmaceutical compositions, and kits.

The present invention provides the combination of:
a. YM53601; and
b. a statin;
   for use in the treatment of a Cryptosporidium infection. The components of the combination may be for separate, simultaneous or sequential administration. The invention extends to corresponding uses, methods of treatment, pharmaceutical compositions, and kits.

The present invention provides the combination of:
a. YM53601; and
b. a statin;
   for use in the treatment of a rotavirus infection. The components of the combination may be for separate, simultaneous or sequential administration. The invention extends to corresponding uses, methods of treatment, pharmaceutical compositions, and kits.

The present invention provides the combination of:
a. YM53601; and
b. a statin;
   for use in the treatment of a Salmonella infection. The components of the combination may be for separate, simultaneous or sequential administration. The invention extends to corresponding uses, methods of treatment, pharmaceutical compositions, and kits.

The present invention provides the combination of:
a. lapaquistat or YM53601; and
b. lovastatin;
   for use in the treatment of an infection by an intracellular pathogen that does not express an endogenous squalene synthase. The components of the combination may be for separate, simultaneous or sequential administration. The invention extends to corresponding uses, methods of treatment, pharmaceutical compositions, and kits.

The present invention provides the combination of:
a. lapaquistat or YM53601; and
b. lovastatin; and
   for use in the treatment of an intracellular parasitic infection by a parasite that does not express an endogenous squalene synthase. The components of the combination may be for separate, simultaneous or sequential administration. The invention extends to corresponding uses, methods of treatment, pharmaceutical compositions, and kits.

The present invention provides the combination of:
a. lapaquistat or YM53601; and
b. lovastatin;
   for use in the treatment of a Cryptosporidium infection. The components of the combination may be for separate, simultaneous or sequential administration. The invention extends to corresponding uses, methods of treatment, pharmaceutical compositions, and kits.

The present invention provides the combination of:
a. lapaquistat or YM53601; and
b. lovastatin;
   for use in the treatment of a rotavirus infection. The components of the combination may be for separate, simultaneous or sequential administration. The invention extends to corresponding uses, methods of treatment, pharmaceutical compositions, and kits.

The present invention provides the combination of:
a. lapaquistat or YM53601; and
b. lovastatin;
   for use in the treatment of a Salmonella infection. The components of the combination may be for separate, simultaneous or sequential administration. The invention extends to corresponding uses, methods of treatment, pharmaceutical compositions, and kits.

The present invention provides the combination of:
a. lapaquistat; and
b. lovastatin;
   for use in the treatment of a Cryptosporidium infection. The components of the combination may be for separate, simultaneous or sequential administration. The invention extends to corresponding uses, methods of treatment, pharmaceutical compositions, and kits.

The present invention provides the combination of:
a. lapaquistat; and
b. lovastatin; and
   for use in the treatment of a rotavirus infection. The components of the combination may be for separate, simultaneous or sequential administration. The invention extends to corresponding uses, methods of treatment, pharmaceutical compositions, and kits.

The present invention provides the combination of:
a. lapaquistat; and
b. lovastatin;
   for use in the treatment of a Salmonella infection. The components of the combination may be for separate, simultaneous or sequential administration. The invention extends to corresponding uses, methods of treatment, pharmaceutical compositions, and kits.

The present invention provides the combination of:
a. YM53601; and
b. lovastatin; and
   for use in the treatment of a Cryptosporidium infection. The components of the combination may be for separate, simultaneous or sequential administration. The invention extends to corresponding uses, methods of treatment, pharmaceutical compositions, and kits.

The present invention provides the combination of:
a. YM53601; and
b. lovastatin;
   for use in the treatment of a rotavirus infection. The components of the combination may be for separate, simultaneous or sequential administration. The invention extends to corresponding uses, methods of treatment, pharmaceutical compositions, and kits.

The present invention provides the combination of:
a. YM53601; and
b. lovastatin;
   for use in the treatment of a Salmonella infection. The components of the combination may be for separate, simultaneous or sequential administration. The invention extends to corresponding uses, methods of treatment, pharmaceutical compositions, and kits.

The present invention provides the combination of:
a. lapaquistat or YM53601; and
b. atorvastatin;
   for use in the treatment of an infection by an intracellular pathogen that does not express an endogenous squalene synthase. The components of the combination may be for separate, simultaneous or sequential administration. The invention extends to corresponding uses, methods of treatment, pharmaceutical compositions, and kits.

The present invention provides the combination of:
a. lapaquistat or YM53601; and
b. atorvastatin; and
   for use in the treatment of a parasitic infection by an intracellular parasite that does not express an endogenous squalene synthase. The components of the combination may be for separate, simultaneous or sequential administration. The invention extends to corresponding uses, methods of treatment, pharmaceutical compositions, and kits.

The present invention provides the combination of:
a. lapaquistat or YM53601; and
b. atorvastatin;
   for use in the treatment of a Cryptosporidium infection. The components of the combination may be for separate, simultaneous or sequential administration. The invention extends to corresponding uses, methods of treatment, pharmaceutical compositions, and kits.

The present invention provides the combination of:
a. lapaquistat or YM53601; and
b. atorvastatin;
   for use in the treatment of a rotavirus infection. The components of the combination may be for separate, simultaneous or sequential administration. The invention extends to corresponding uses, methods of treatment, pharmaceutical compositions, and kits.

The present invention provides the combination of:
a. lapaquistat or YM53601; and
b. atorvastatin;
   for use in the treatment of a Salmonella infection. The components of the combination may be for separate, simultaneous or sequential administration. The invention extends to corresponding uses, methods of treatment, pharmaceutical compositions, and kits.

The present invention provides the combination of:
a. lapaquistat; and
b. atorvastatin;
   for use in the treatment of a Cryptosporidium infection. The components of the combination may be for separate, simultaneous or sequential administration. The invention extends to corresponding uses, methods of treatment, pharmaceutical compositions, and kits.

The present invention provides the combination of:
a. lapaquistat; and
b. atorvastatin; and
   for use in the treatment of a rotavirus infection. The components of the combination may be for separate, simultaneous or sequential administration. The invention extends to corresponding uses, methods of treatment, pharmaceutical compositions, and kits.

The present invention provides the combination of:
a. lapaquistat; and
b. atorvastatin;
   for use in the treatment of a Salmonella infection. The components of the combination may be for separate, simultaneous or sequential administration. The invention extends to corresponding uses, methods of treatment, pharmaceutical compositions, and kits.

The present invention provides the combination of:
a. YM53601; and
b. atorvastatin; and
   for use in the treatment of a Cryptosporidium infection. The components of the combination may be for separate, simultaneous or sequential administration. The invention extends to corresponding uses, methods of treatment, pharmaceutical compositions, and kits.

The present invention provides the combination of:
a. YM53601; and
b. atorvastatin;
   for use in the treatment of a rotavirus infection. The components of the combination may be for separate, simultaneous or sequential administration. The invention extends to corresponding uses, methods of treatment, pharmaceutical compositions, and kits.

The present invention provides the combination of:
a. YM53601; and
b. atorvastatin;
   for use in the treatment of a Salmonella infection. The components of the combination may be for separate, simultaneous or sequential administration. The invention extends to corresponding uses, methods of treatment, pharmaceutical compositions, and kits.

### FDFT1 Inhibitors and Other Anti-Parasitic Agents

The present invention provides an FDFT1 inhibitor for use in the treatment of an infection by an intracellular pathogen that does not express an endogenous squalene synthase. The FDFT1 inhibitor may be combined with nitazoxanide, paromomycin or halofuginone (e.g. nitazoxanide) for simultaneous, separate or sequential administration. The invention extends to corresponding methods of treatment, pharmaceutical compositions, combinations, and kits.

The present invention provides lapaquistat or YM53601 for use in the treatment of an infection by an intracellular pathogen that does not express an endogenous squalene synthase. The lapaquistat or YM53601 may be combined with nitazoxanide, paromomycin or halofuginone (e.g. nitazoxanide) for simultaneous, separate or sequential administration. The invention extends to corresponding methods of treatment, pharmaceutical compositions, combinations, and kits.

The present invention provides lapaquistat or YM53601 for use in the treatment of a parasitic infection by an intracellular parasite that does not express an endogenous squalene synthase. The lapaquistat or YM53601 may be combined with nitazoxanide, paromomycin or halofuginone (e.g. nitazoxanide) for simultaneous, separate or sequential administration. The invention extends to corresponding methods of treatment, pharmaceutical compositions, combinations, and kits.

The present invention provides lapaquistat or YM53601 for use in the treatment of a Cryptosporidium infection. The lapaquistat or YM53601 may be combined with a statin for simultaneous, separate or sequential administration. The invention extends to corresponding methods of treatment, pharmaceutical compositions, combinations, and kits.

The present invention provides lapaquistat for use in the treatment of an infection by an intracellular pathogen that does not express an endogenous squalene synthase. The lapaquistat may be combined with nitazoxanide, paromomycin or halofuginone (e.g. nitazoxanide) for simultaneous, separate or sequential administration. The invention extends to corresponding methods of treatment, pharmaceutical compositions, combinations, and kits.

The present invention provides lapaquistat for use in the treatment of an intracellular parasitic infection by a parasite that does not express an endogenous squalene synthase. The lapaquistat may be combined with nitazoxanide, paromomycin or halofuginone (e.g. nitazoxanide) for simultaneous, separate or sequential administration. The invention extends to corresponding methods of treatment, pharmaceutical compositions, combinations, and kits.

The present invention provides lapaquistat for use in the treatment of a Cryptosporidium infection. The lapaquistat may be combined with nitazoxanide, paromomycin or halofuginone (e.g. nitazoxanide) for simultaneous, separate or sequential administration. The invention extends to corresponding methods of treatment, pharmaceutical compositions, combinations, and kits.

The present invention provides YM53601 for use in the treatment of an infection by an intracellular pathogen that does not express an endogenous squalene synthase. The YM53601 may be combined with nitazoxanide, paromomycin or halofuginone (e.g. nitazoxanide) for simultaneous, separate or sequential administration. The invention extends to corresponding methods of treatment, pharmaceutical compositions, combinations, and kits.

The present invention provides YM53601 for use in the treatment of a parasitic infection by an intracellular parasite that does not express an endogenous squalene synthase. The YM53601 may be combined with nitazoxanide, paromomycin or halofuginone (e.g. nitazoxanide) for simultaneous, separate or sequential administration. The invention extends to corresponding methods of treatment, pharmaceutical compositions, combinations, and kits.

The present invention provides YM53601 for use in the treatment of a Cryptosporidium infection. The YM53601 may be combined with nitazoxanide, paromomycin or halofuginone (e.g. nitazoxanide) for simultaneous, separate or sequential administration. The invention extends to corresponding methods of treatment, pharmaceutical compositions, combinations, and kits.

The present invention provides the combination of:
a. an FDFT1 inhibitor; and
b. nitazoxanide, paromomycin or halofuginone (e.g. nitazoxanide);
   for use in the treatment of an infection by an intracellular pathogen that does not express an endogenous squalene synthase. The components of the combination may be for separate, simultaneous or sequential administration. The invention extends to corresponding uses, methods of treatment, pharmaceutical compositions, and kits.

The present invention provides the combination of:
a. lapaquistat or YM53601; and
b. nitazoxanide, paromomycin or halofuginone (e.g. nitazoxanide);
   for use in the treatment of an infection by an intracellular pathogen that does not express an endogenous squalene synthase. The components of the combination may be for separate, simultaneous or sequential administration. The invention extends to corresponding uses, methods of treatment, pharmaceutical compositions, and kits.

The present invention provides the combination of:
a. lapaquistat or YM53601; and
b. nitazoxanide, paromomycin or halofuginone (e.g. nitazoxanide);
   for use in the treatment of an intracellular parasitic infection by a parasite that does not express an endogenous squalene synthase. The components of the combination may be for separate, simultaneous or sequential administration. The invention extends to corresponding uses, methods of treatment, pharmaceutical compositions, and kits.

The present invention provides the combination of:
a. lapaquistat or YM53601; and
b. nitazoxanide, paromomycin or halofuginone (e.g. nitazoxanide);
   for use in the treatment of a Cryptosporidium infection. The components of the combination may be for separate, simultaneous or sequential administration. The invention extends to corresponding uses, methods of treatment, pharmaceutical compositions, and kits.

The present invention provides the combination of:
a. lapaquistat or YM53601; and
b. nitazoxanide, paromomycin or halofuginone (e.g. nitazoxanide);
   for use in the treatment of a rotavirus infection. The components of the combination may be for separate, simultaneous or sequential administration. The invention extends to corresponding uses, methods of treatment, pharmaceutical compositions, and kits.

The present invention provides the combination of:
a. lapaquistat or YM53601; and
b. nitazoxanide, paromomycin or halofuginone (e.g. nitazoxanide);
   for use in the treatment of a Salmonella infection. The components of the combination may be for separate, simultaneous or sequential administration. The invention extends to corresponding uses, methods of treatment, pharmaceutical compositions, and kits.

The present invention provides the combination of:
a. lapaquistat; and
b. nitazoxanide, paromomycin or halofuginone (e.g. nitazoxanide);
   for use in the treatment of a Cryptosporidium infection. The components of the combination may be for separate, simultaneous or sequential administration. The invention extends to corresponding uses, methods of treatment, pharmaceutical compositions, and kits.

The present invention provides the combination of:
a. lapaquistat; and
b. nitazoxanide, paromomycin or halofuginone (e.g. nitazoxanide);
   for use in the treatment of a rotavirus infection. The components of the combination may be for separate, simultaneous or sequential administration. The invention extends to corresponding uses, methods of treatment, pharmaceutical compositions, and kits.

The present invention provides the combination of:
a. lapaquistat; and
b. nitazoxanide, paromomycin or halofuginone (e.g. nitazoxanide);
   for use in the treatment of a Salmonella infection. The components of the combination may be for separate, simultaneous or sequential administration. The invention extends to corresponding uses, methods of treatment, pharmaceutical compositions, and kits.

The present invention provides the combination of:
a. YM53601; and
b. nitazoxanide, paromomycin or halofuginone (e.g. nitazoxanide);
   for use in the treatment of a Cryptosporidium infection. The components of the combination may be for separate, simultaneous or sequential administration. The invention extends to corresponding uses, methods of treatment, pharmaceutical compositions, and kits.

The present invention provides the combination of:
a. YM53601; and
b. nitazoxanide, paromomycin or halofuginone (e.g. nitazoxanide);
   for use in the treatment of a rotavirus infection. The components of the combination may be for separate, simultaneous or sequential administration. The invention extends to corresponding uses, methods of treatment, pharmaceutical compositions, and kits.

The present invention provides the combination of:
a. YM53601; and
b. nitazoxanide, paromomycin or halofuginone (e.g. nitazoxanide);
   for use in the treatment of a Salmonella infection. The components of the combination may be for separate, simultaneous or sequential administration. The invention extends to corresponding uses, methods of treatment, pharmaceutical compositions, and kits.

The present invention provides the combination of:
a. lapaquistat or YM53601; and
b. nitazoxanide;
   for use in the treatment of a Cryptosporidium infection. The components of the combination may be for separate, simultaneous or sequential administration. The invention extends to corresponding uses, methods of treatment, pharmaceutical compositions, and kits.

The present invention provides the combination of:
a. lapaquistat or YM53601; and
b. nitazoxanide;
   for use in the treatment of a rotavirus infection. The components of the combination may be for separate, simultaneous or sequential administration. The invention extends to corresponding uses, methods of treatment, pharmaceutical compositions, and kits.

The present invention provides the combination of:
a. lapaquistat or YM53601; and
b. nitazoxanide;
   for use in the treatment of a Salmonella infection. The components of the combination may be for separate, simultaneous or sequential administration. The invention extends to corresponding uses, methods of treatment, pharmaceutical compositions, and kits.

The present invention provides the combination of:
a. lapaquistat; and
b. nitazoxanide;
   for use in the treatment of a Cryptosporidium infection. The components of the combination may be for separate, simultaneous or sequential administration. The invention extends to corresponding uses, methods of treatment, pharmaceutical compositions, and kits.

The present invention provides the combination of:
a. lapaquistat; and
b. nitazoxanide;
   for use in the treatment of a rotavirus infection. The components of the combination may be for separate, simultaneous or sequential administration. The invention extends to corresponding uses, methods of treatment, pharmaceutical compositions, and kits.

The present invention provides the combination of:
a. lapaquistat; and
b. nitazoxanide;
   for use in the treatment of a Salmonella infection. The components of the combination may be for separate, simultaneous or sequential administration. The invention extends to corresponding uses, methods of treatment, pharmaceutical compositions, and kits.

The present invention provides the combination of:
a. YM53601; and
b. nitazoxanide;
   for use in the treatment of a Cryptosporidium infection. The components of the combination may be for separate, simultaneous or sequential administration. The invention extends to corresponding uses, methods of treatment, pharmaceutical compositions, and kits.

The present invention provides the combination of:
a. YM53601; and
b. nitazoxanide;
   for use in the treatment of a rotavirus infection. The components of the combination may be for separate, simultaneous or sequential administration. The invention extends to corresponding uses, methods of treatment, pharmaceutical compositions, and kits.

The present invention provides the combination of:
a. YM53601; and
b. nitazoxanide;
   for use in the treatment of a Salmonella infection. The components of the combination may be for separate, simultaneous or sequential administration. The invention extends to corresponding uses, methods of treatment, pharmaceutical compositions, and kits.

### Triple Combinations

The present invention provides the combination of:
a. a FDFT1 inhibitor;
b. a statin; and
c. one or more other drugs used in the treatment or prophylaxis of a parasitic infection;
   for use in the treatment of a Cryptosporidium infection. The invention extends to corresponding uses, methods of treatment, pharmaceutical compositions, and kits.

The present invention provides the combination of:
a. a FDFT1 inhibitor;
b. a statin; and
c. nitazoxanide, paromomycin or halofuginone;
   for use in the treatment of a Cryptosporidium infection. The invention extends to corresponding uses, methods of treatment, pharmaceutical compositions, and kits.

The present invention provides the combination of:
a. lapaquistat or YM53601;
b. a statin; and
c. nitazoxanide, paromomycin or halofuginone;
   for use in the treatment of a Cryptosporidium infection. The invention extends to corresponding uses, methods of treatment, pharmaceutical compositions, and kits.

The present invention provides the combination of:
a. lapaquistat or YM53601;
b. a statin; and
c. nitazoxanide;
   for use in the treatment of a Cryptosporidium infection. The invention extends to corresponding uses, methods of treatment, pharmaceutical compositions, and kits.

The present invention provides the combination of:
a. lapaquistat;
b. a statin; and
c. nitazoxanide;
   for use in the treatment of a Cryptosporidium infection. The invention extends to corresponding uses, methods of treatment, pharmaceutical compositions, and kits.

The present invention provides the combination of:
a. YM53601;
b. a statin; and
c. nitazoxanide;
   for use in the treatment of a Cryptosporidium infection. The invention extends to corresponding uses, methods of treatment, pharmaceutical compositions, and kits.

The present invention provides the combination of:
a. lapaquistat or YM53601;
b. lovastatin; and
c. nitazoxanide, paromomycin or halofuginone;
   for use in the treatment of a Cryptosporidium infection. The invention extends to corresponding uses, methods of treatment, pharmaceutical compositions, and kits.

The present invention provides the combination of:
a. lapaquistat or YM53601;
b. lovastatin; and
c. nitazoxanide;
   for use in the treatment of a Cryptosporidium infection. The invention extends to corresponding uses, methods of treatment, pharmaceutical compositions, and kits.

The present invention provides the combination of:
a. lapaquistat;
b. lovastatin; and
c. nitazoxanide;
   for use in the treatment of a Cryptosporidium infection. The invention extends to corresponding uses, methods of treatment, pharmaceutical compositions, and kits.

The present invention provides the combination of:
a. YM53601;
b. lovastatin; and
c. nitazoxanide;
   for use in the treatment of a Cryptosporidium infection. The invention extends to corresponding uses, methods of treatment, pharmaceutical compositions, and kits.

The present invention provides the combination of:
a. lapaquistat or YM53601;
b. atorvastatin; and
c. nitazoxanide, paromomycin or halofuginone;
   for use in the treatment of a Cryptosporidium infection. The invention extends to corresponding uses, methods of treatment, pharmaceutical compositions, and kits.

The present invention provides the combination of:
a. lapaquistat or YM53601;
b. atorvastatin; and
c. nitazoxanide;
   for use in the treatment of a Cryptosporidium infection. The invention extends to corresponding uses, methods of treatment, pharmaceutical compositions, and kits.

The present invention provides the combination of:
a. lapaquistat;
b. atorvastatin; and
c. nitazoxanide;
   for use in the treatment of a Cryptosporidium infection. The invention extends to corresponding uses, methods of treatment, pharmaceutical compositions, and kits.

The present invention provides the combination of:
a. YM53601;
b. atorvastatin; and
c. nitazoxanide;
   for use in the treatment of a Cryptosporidium infection. The invention extends to corresponding uses, methods of treatment, pharmaceutical compositions, and kits.

### EXAMPLES

### Example 1—Microscopy-Based Whole Genome CRISPR-Cas9 Screen for Host Genes Affecting Cryptosporidium Growth In Vitro

HCT-8 cells (ATCC; CCL-244) were transformed using the Genome-CRISP Inducible Cas9 human AAVS1 Safe Harbour knock-in kit (Cat. No. SH016) to create a cell line capable of doxycycline-inducible Cas9 expression. A single clone showing the most efficient gene knockdown upon CRISPR guide RNA transfection with doxycycline treatment was selected. 200 nM of crRNA guides from a full human genome library (Dharmacon Human Edit-R-Genome; Cat. No. GP/GC-005005-xx) was mixed in a 1:1 ratio with tracrRNA and transferred to 384-well plates. Each well contained four crRNA guides targeting a single gene. Each plate also contained non-targeting crRNA guides, as well as positive controls corresponding to gene targets previously identified as reducing and increasing Cryptosporidium growth. Plates were sealed and frozen at −20° C. until use.

Plates were thawed at room temperature and transfectant (Dharmafect2; Cat. No. T-2002-03, dissolved in Opti-MEM) was dispensed into each well. Cas9-HCT-8 cells were then seeded into all wells (2250 cells/well) in media containing doxycycline (RPMI with 10% FCS). Plates were incubated at 37° C., 5% CO₂ for 72 hours. Cryptosporidium parvum oocysts (Bunchgrass Farms; Dreary Idaho, USA) were prepared for infection by bleach treating (1% sodium hypochlorite) on ice for 5 minutes to remove bacterial contaminants, followed by a wash step and incubation in 0.75% sodium taurocholate for 10 minutes at 37° C. to trigger excystation. Oocysts were resuspended in infection media (RPMI with 1% FCS) containing doxycycline and dispensed into all wells (12,500 oocysts/well). Plates were incubated at 37° C., 5% CO₂ for 49 hours.

Plates were fixed by addition of concentrated paraformaldehyde (PFA) to each well to produce a final concentration of 4% PFA. 20 minutes post-fixing, plates were washed three times followed by permeabilisation (0.25% Triton X-100 in PBS) for 10 minutes and blocking (4% BSA in PBS) for 1 hour. Cells were stained with a Cryptosporidium female-specific antibody (rat anti-COWP1) overnight in 1% BSA. The next day, plates were washed in PBS and incubated with a fluorescently-conjugated lectin that detects all Cryptosporidium parasites (VVL-Fluorescein, VectorLabs; Cat. No. FL-1231-2), an anti-rat antibody conjugated to AlexaFLuor-647, AlexaFluor-546-conjugated to phalloidin to detect host cell actin, and Hoechst to detect host cells. Plates were incubated for 90 minutes, washed in PBS, and sealed.

An Opera Phenix high-content screening system (PerkinElmer) was used to image all plates to image host cells, host actin, parasites, and any females among those parasites. 5 fields of view per well were captured. Harmony high-content imaging and analysis software (PerkinElmer) was used to analyse all images and output information in the form of host and parasite numbers, numbers of females, host actin recruitment to parasites, and size information on each parasite. The resulting information was subjected to normalisation algorithms to account for any plate edge effects across all 183 384-well plates used in the experiment.

### Example 2—In Vitro IC50 Curves with Lapaquistat

HCT-cells (ATCC; CCL-244) were seeded in a 96-well plate at a density of 30,000 cells/well in complete media (RPMI with 10% FCS) and allowed to grow for 48 hours. Cryptosporidium parvum oocysts (Bunchgrass Farms; Dreary Idaho, USA) were prepared for infection by bleach treating (1% sodium hypochlorite) on ice for 5 minutes to remove bacterial contaminants, followed by a wash step and incubation in 0.75% sodium taurocholate for 10 minutes at 37° C. to trigger excystation. Oocysts were resuspended in infection media (RPMI with 1% FCS) and added to cells in 100 uL media (20,000 oocysts/well).

A 10 mM solution of lapaquistat acetate (MedChem Express; Cat. No. HY-16274-1 mg) was created using DMSO as the solvent. From this stock, a half-logarithmic (3.16-fold) dilution series was created in infection media, at 2× the intended final concentration. 3 hours post-infection, 100 uL of each drug dilution was added to the wells. Each drug concentration comprised 6 technical replicates. The plate was incubated at 37° C., 5% CO₂ for a total of 49 hours post-infection.

Cells were fixed in 4% paraformaldehyde for 20 minutes, followed by washing three times in PBS. Cells were permeabilised (0.25% Triton X-100 in PBS) for 10 minutes and blocked (4% BSA in PBS) for 1 hour. Cells were then incubated with a Cryptosporidium female-specific antibody (rat anti-COWP1) overnight in 1% BSA. The next day, the plate was washed in PBS and incubated with a fluorescently-conjugated lectin that detects all Cryptosporidium parasites (VVL-Fluorescein, VectorLabs; Cat. No. FL-1231-2), an anti-rat antibody conjugated to AlexaFLuor-647, and Hoechst to detect host cells. The plate was stained for 90 minutes and washed in PBS. The plate was then imaged using a Cytation cell imaging multimode reader (BioTek) to count host nuclei, total number of parasites, and the number of female parasites among them.

### Example 3—Results of Lapaquistat Treatment of Cryptosporidium Infected Cells

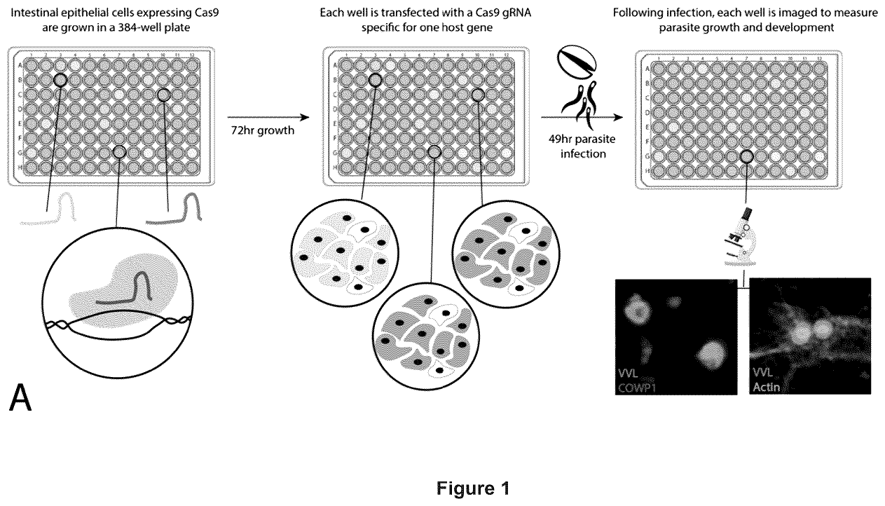

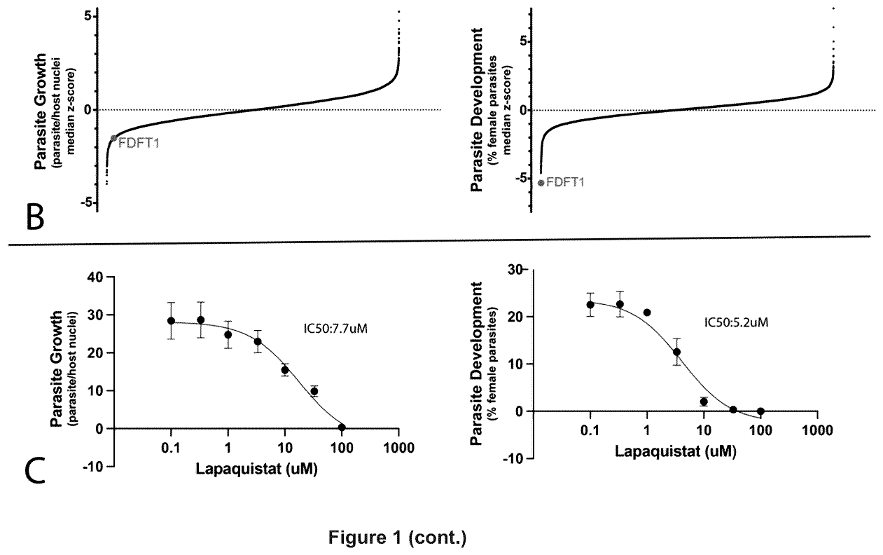

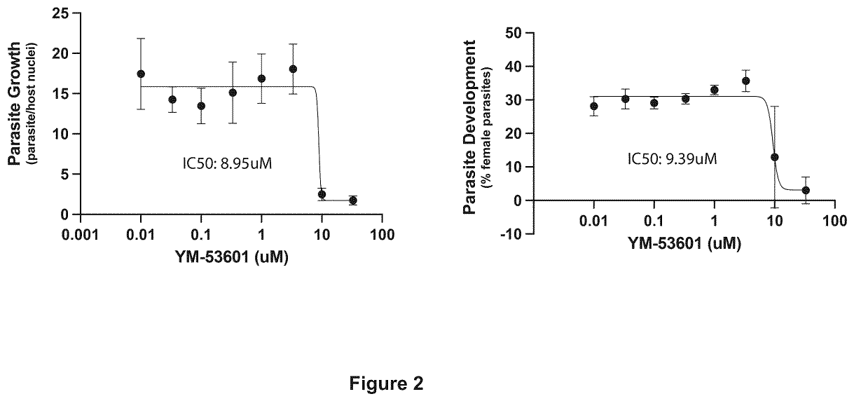

The Cryptosporidium parasite is an obligate intracellular parasite and relies on the host cell for survival. In order to identify the host genes required for Cryptosporidium growth and development we developed a high-throughput microscopy-based CRISPR assay that measures parasite number, size, and development in human intestinal epithelial cells (HCT-8 adenocarcinoma) (FIG. 1A). In vitro, Cryptosporidium replicates within cells and develops into sexual stages—male and female. We were able to detect and quantify this development using an antibody that is specific for female parasites.

Through this screen we found the cholesterol biosynthesis pathway is required for growth and development of the Cryptosporidium parasite, particularly the gene responsible for squalene synthesis, FDFT1 (FIG. 1B). FDFT1 is a rate limiting step in the cholesterol biosynthesis pathway and TAK-475 or lapaquistat is a highly specific inhibitor for FDFT1, originally developed to treat hypercholesterolemia [6,7,8]. Treatment of Cryptosporidium infected intestinal cell monolayers demonstrated that lapaquistat is effective in blocking both parasite growth (IC50 7.7 uM) and development (IC50 5.2 uM) in vitro (FIG. 1C).

Lapaquistat was originally developed for the treatment of hypercholesteremia by Takeda Pharmaceuticals, but was eventually abandoned after Phase 3 clinical trials when it was found that patients in the high dose group demonstrated elevated liver enzymes after 8 weeks of treatment. Lapaquistat has shown efficacy against Cryptosporidium and because this drug already has substantial safety data (>3 large clinical trials) we believe there is strong potential for repurposing Lapaquistat for Cryptosporidium treatment. Cryptosporidium treatment would not require chronic dosing and because lapaquistat has low bioavailability we believe the drug will concentrate in the intestine—at the site of infection—and increase potency allowing for a low dose to be used [7,8]. Furthermore, because many intracellular pathogens (including viruses and bacteria) require cholesterol biosynthesis within their infected host cell, we believe that inhibition of FDFT1 could be used a broad-spectrum treatment for intestinal infections.

### Example 4—Results of YM-53601 Treatment of Cryptosporidium Infected Cells

HCT-cells (ATCC; CCL-244) were seeded in a 96-well plate at a density of 30,000 cells/well in complete media (RPMI with 10% FCS) and allowed to grow for 48 hours.

Cryptosporidium parvum oocysts (Bunchgrass Farms; Dreary Idaho, USA) were prepared for infection by bleach treating (1% sodium hypochlorite) on ice for 5 minutes to remove bacterial contaminants, followed by a wash step and incubation in 0.75% sodium taurocholate for 10 minutes at 37° C. to trigger excystation. Oocysts were resuspended in infection media (RPMI with 1% FCS) and added to cells in 100 uL media (20,000 oocysts/well).

A 10 mM solution of YM-53601 (AdooQ Bioscience, reference A13483) was created using DMSO as the solvent. From this stock, a half-logarithmic (3.16-fold) dilution series was created in infection media, at 2× the intended final concentration. 3 hours post-infection, 100 uL of each drug dilution was added to the wells. Each drug concentration comprised 6 technical replicates. The plate was incubated at 37° C., 5% CO2 for a total of 49 hours post-infection.

Cells were fixed in 4% paraformaldehyde for 20 minutes, followed by washing three times in PBS. Cells were permeabilised (0.25% Triton X-100 in PBS) for 10 minutes and blocked (4% BSA in PBS) for 1 hour. Cells were then incubated with a Cryptosporidium female-specific antibody (rat anti-COWP1) overnight in 1% BSA. The next day, the plate was washed in PBS and incubated with a fluorescently-conjugated lectin that detects all Cryptosporidium parasites (VVL-Fluorescein, VectorLabs; Cat. No. FL-1231-2), an anti-rat antibody conjugated to AlexaFLuor-647, and Hoechst to detect host cells. The plate was stained for 90 minutes and washed in PBS.

The plate was then imaged using a Cytation cell imaging multimode reader (BioTek) to count host nuclei, total number of parasites, and the number of female parasites among them.

### Example 5—Lapaquistat Combines with Statins Synergistically

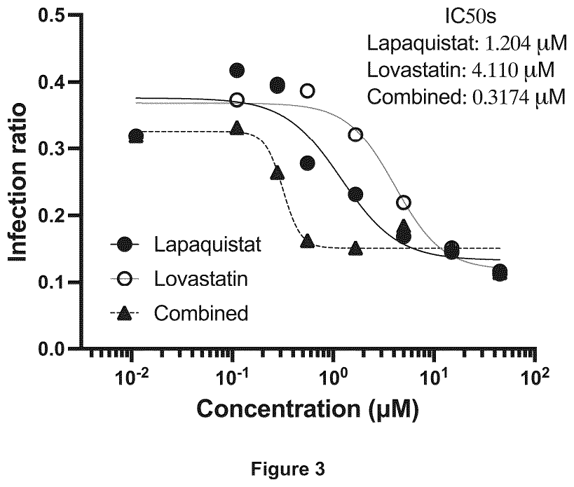

Intestinal epithelial cells (HCT-8) were infected with Cryptosporidium parasites and treated with increasing concentrations of Lapaquistat acetate or Lovastatin, either alone or in combination. After 48 hours of infection, cells were then fixed and stained with a lectin that specifically marks parasites and a Hoechst dye that specifically labels DNA. Cells and parasites were then visualized with an Opera Phenix imaging microscope and infection ratio was calculated as number of parasites/number of host cells. We found that the potency of Lapaquistat, with respect to its ability to limit Cryptosporidium growth, was synergistically increased (as defined in Meyer et al., PMID 30797775) when in combination with Lovastatin (FIG. 3). In this regard, the IC50s for Lapaquistat alone, Lovastatin alone and the combination were 1.2 μM, 4.1 μM and 0.32 μM. Hence, Lapaquistat acts in a synergistic manner in combination with statins against Cryptosporidium infection.

### EQUIVALENTS AND SCOPE

Those skilled in the art will appreciate that the present invention is defined by the appended claims and not by the Examples or other description of certain embodiments included herein.

Similarly, the singular forms “a”, “an”, and “the” include plural referents unless the context clearly dictates otherwise.

Unless defined otherwise above, all technical and scientific terms used herein have the same meaning as commonly understood by one of ordinary skill in the art to which this invention belongs. Any methods and materials similar or equivalent to those described herein can also be used in the practice or testing of the present invention. Generally, nomenclatures used in connection with, and techniques of, cell and tissue culture, molecular biology, immunology, genetics and protein and nucleic acid chemistry described herein are those well-known and commonly used in the art, or according to manufacturer's specifications.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### EMBODIMENTS

The present invention also provides the embodiments described below:
1. A method of treating or preventing an infection, comprising administering to a patient in need thereof a therapeutically or prophylactically effective amount of a farnesyl-diphosphate farnesyltransferase 1 (FDFT1) inhibitor, or a pharmaceutically acceptable salt thereof.
2. The method of embodiment 1, wherein the infection is an infection by a pathogen which is incapable of endogenous cholesterol biosynthesis.
3. The method of embodiment 1 or 2, wherein the infection is an infection by a pathogen which is incapable of endogenous squalene synthesis.
4. The method of any preceding embodiment, wherein, the infection is an infection by a pathogen which does not express an endogenous squalene synthase.
5. The method of any preceding embodiment, wherein the infection is a parasitic, viral or bacterial infection.
6. The method of any preceding embodiment, wherein the infection is a Cryptosporidium infection.
7. The method of any one of embodiments 1 to 5, wherein the infection is a rotavirus infection.
8. The method of any one of embodiments 1 to 5, wherein the infection is a Salmonella infection.
9. A method of treating or preventing a parasitic infection, comprising administering to a patient in need thereof a therapeutically or prophylactically effective amount of a farnesyl-diphosphate farnesyltransferase 1 (FDFT1) inhibitor, or a pharmaceutically acceptable salt thereof.
10. A method of treating or preventing a Cryptosporidium infection, comprising administering to a patient in need thereof a therapeutically or prophylactically effective amount of a farnesyl-diphosphate farnesyltransferase 1 (FDFT1) inhibitor, or a pharmaceutically acceptable salt thereof.
11. A method of treating or preventing a viral infection, comprising administering to a patient in need thereof a therapeutically or prophylactically effective amount of a farnesyl-diphosphate farnesyltransferase 1 (FDFT1) inhibitor, or a pharmaceutically acceptable salt thereof.
12. A method of treating or preventing a rotavirus infection, comprising administering to a patient in need thereof a therapeutically or prophylactically effective amount of a farnesyl-diphosphate farnesyltransferase 1 (FDFT1) inhibitor, or a pharmaceutically acceptable salt thereof.
13. A method of treating or preventing a bacterial infection, comprising administering to a patient in need thereof a therapeutically or prophylactically effective amount of a farnesyl-diphosphate farnesyltransferase 1 (FDFT1) inhibitor, or a pharmaceutically acceptable salt thereof.
14. A method of treating or preventing a Salmonella infection, comprising administering to a patient in need thereof a therapeutically or prophylactically effective amount of a farnesyl-diphosphate farnesyltransferase 1 (FDFT1) inhibitor, or a pharmaceutically acceptable salt thereof.
15. A farnesyl-diphosphate farnesyltransferase 1 (FDFT1) inhibitor, or a pharmaceutically acceptable salt thereof, for use in a method of treating or preventing an infection.
16. The farnesyl-diphosphate farnesyltransferase 1 (FDFT1) inhibitor, or a pharmaceutically acceptable salt thereof, for use according to embodiment 15, wherein the infection is an infection by a pathogen which is incapable of endogenous cholesterol biosynthesis.
17. The farnesyl-diphosphate farnesyltransferase 1 (FDFT1) inhibitor, or a pharmaceutically acceptable salt thereof, for use according to embodiment 15 or 16, wherein the infection is an infection by a pathogen which is incapable of endogenous squalene synthesis.
18. The farnesyl-diphosphate farnesyltransferase 1 (FDFT1) inhibitor, or a pharmaceutically acceptable salt thereof, for use according to any one of embodiments 15 to 17, wherein, the infection is an infection by a pathogen which does not express an endogenous squalene synthase.
19. The farnesyl-diphosphate farnesyltransferase 1 (FDFT1) inhibitor, or a pharmaceutically acceptable salt thereof, for use according to any one of embodiments 15 to 18, wherein the infection is a parasitic, viral or bacterial infection.
20. The farnesyl-diphosphate farnesyltransferase 1 (FDFT1) inhibitor, or a pharmaceutically acceptable salt thereof, for use according to embodiment 19, wherein the infection is a Cryptosporidium infection.
21. The farnesyl-diphosphate farnesyltransferase 1 (FDFT1) inhibitor, or a pharmaceutically acceptable salt thereof, for use according to embodiment 19, wherein the infection is a rotavirus infection.
22. The farnesyl-diphosphate farnesyltransferase 1 (FDFT1) inhibitor, or a pharmaceutically acceptable salt thereof, for use according to embodiment 19, wherein the infection is a Salmonella infection.
23. A farnesyl-diphosphate farnesyltransferase 1 (FDFT1) inhibitor, or a pharmaceutically acceptable salt thereof for use in a method of treating or preventing a parasitic infection.
24. A farnesyl-diphosphate farnesyltransferase 1 (FDFT1) inhibitor, or a pharmaceutically acceptable salt thereof for use in a method of treating or preventing a Cryptosporidium infection.
25. A farnesyl-diphosphate farnesyltransferase 1 (FDFT1) inhibitor, or a pharmaceutically acceptable salt thereof for use in a method of treating or preventing a viral infection.
26. A farnesyl-diphosphate farnesyltransferase 1 (FDFT1) inhibitor, or a pharmaceutically acceptable salt thereof for use in a method of treating or preventing a rotavirus infection.
27. A farnesyl-diphosphate farnesyltransferase 1 (FDFT1) inhibitor, or a pharmaceutically acceptable salt thereof for use in a method of treating or preventing a bacterial infection.
28. A farnesyl-diphosphate farnesyltransferase 1 (FDFT1) inhibitor, or a pharmaceutically acceptable salt thereof for use in a method of treating or preventing a Salmonella infection.
29. The method according to any one of embodiments 1-14 or the FDFT1 inhibitor or pharmaceutically acceptable salt thereof for use according to embodiments 15-28, wherein the FDFT1 inhibitor is lapaquistat, or a pharmaceutically acceptable salt thereof.
30. The method according to any one according to embodiments 1-14 or the FDFT1 inhibitor or pharmaceutically acceptable salt thereof for use according to embodiments 15-28, wherein the FDFT1 inhibitor has the following structure:

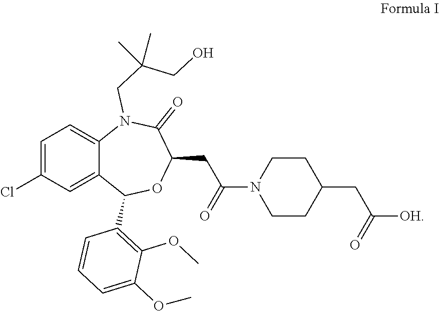 [SMILES: COc1cccc([C@H]2O[C@H](CC(=O)N3CCC(CC(=O)O)CC3)C(=O)N(CC(C)(C)CO)c3ccc(Cl)cc32)c1OC]

31. The method according to any one of embodiments 1-14 or the FDFT1 inhibitor or pharmaceutically acceptable salt thereof for use according to embodiments 15-28, wherein the FDFT1 inhibitor is YM-53601, or a pharmaceutically acceptable salt thereof.
32. The method according to any one according to embodiments 1-14 or the FDFT1 inhibitor or pharmaceutically acceptable salt thereof for use according to embodiments 15-28, wherein the FDFT1 inhibitor has the following structure:

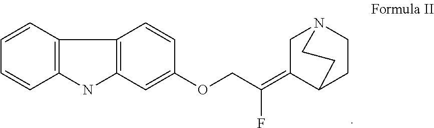 [SMILES: F/C(COc1ccc2c(c1)[nH]c1ccccc12)=C1/CN2CCC1CC2]

33. The method of any one of embodiments 1-14 or 29-32 or the FDFT1 inhibitor or pharmaceutically acceptable salt thereof for use according to any one of embodiments 15-32, wherein the FDFT1 inhibitor is administered simultaneously, sequentially or separately with one or more other drugs used in the treatment or prophylaxis of the parasitic infection or Cryptosporidium infection.
34. The method of any one of embodiments 1-14 or 29-33 or the FDFT1 inhibitor or pharmaceutically acceptable salt thereof for use according to any one of embodiments 15-33, wherein the FDFT1 inhibitor is administered simultaneously, sequentially or separately with one or more other drugs used in the treatment or prophylaxis of the viral infection or rotavirus infection.
35. The method of any one of embodiments 1-14 or 29-34 or the FDFT1 inhibitor or pharmaceutically acceptable salt thereof for use according to any one of embodiments 15-34, wherein the FDFT1 inhibitor is administered simultaneously, sequentially or separately with one or more other drugs used in the treatment or prophylaxis of the bacterial infection or Salmonella infection.
36. The method or FDFT1 inhibitor or pharmaceutically acceptable salt thereof for use according to any one of embodiments 33-35, wherein the one or more other drugs is formulated in a combined preparation with the FDFT1 inhibitor or pharmaceutically acceptable salt thereof.
37. The method or FDFT1 inhibitor or pharmaceutically acceptable salt thereof for use according to any one of embodiments 33-36, wherein the one or more other drugs comprises nitazoxanide, paromomycin or halofuginone.
38. The method or FDFT1 inhibitor or pharmaceutically acceptable salt thereof for use according to any one of embodiments 33-37, wherein the one or more other drugs comprises nitazoxanide.
39. The method of any one of embodiments 1-14 or 29-38 or the FDFT1 inhibitor or pharmaceutically acceptable salt thereof for use according to any one of embodiments 15-38, wherein the FDFT1 inhibitor is administered to the patient orally, intravenously, intramuscularly, intrathecally, subcutaneously, sublingually, buccally, rectally, vaginally, ocularly, oticly, nasally, by inhalation, by nebulization, cutaneously, topically or transdermally.
40. The method of any one of embodiments 1-14 or 29-39 or the FDFT1 inhibitor or pharmaceutically acceptable salt thereof for use according to any one of embodiments 15-39, wherein the one or more other drugs are administered to the patient orally, intravenously, intramuscularly, intrathecally, subcutaneously, sublingually, buccally, rectally, vaginally, ocularly, oticly, nasally, by inhalation, by nebulization, cutaneously, topically or transdermally.
41. The method of any one of embodiments 1-14 or 29-39 or the FDFT1 inhibitor or pharmaceutically acceptable salt thereof for use according to any one of embodiments 15-40, wherein the subject is a human.
42. The method of any one of embodiments 1-14 or 29-40 or the FDFT1 inhibitor or pharmaceutically acceptable salt thereof for use according to any one of embodiments 15-40, wherein the subject is a cow (cattle), a sheep, a goat, a cat, a dog, a pig (swine), a horse or a rabbit.
43. The method of any one of embodiments 1-14 or 29-42 or the FDFT1 inhibitor or pharmaceutically acceptable salt thereof for use according to any one of embodiments 15-42, wherein the FDFT1 inhibitor is administered in a dose sufficient to achieve at least 50%, at least 60%, at least 70%, at least 80%, at least 90%, at least 95% or at least 99% inhibition of FDFT1 activity.
44. The method of any one of embodiments 1-14 or 29-43 or the FDFT1 inhibitor or pharmaceutically acceptable salt thereof for use according to any one of embodiments 15-43, wherein the FDFT1 inhibitor is administered in a dose sufficient to achieve at least 50%, at least 60%, at least 70%, at least 80%, at least 90%, at least 95% or at least 99% inhibition of squalene production in a target cell.
45. The method of any one of embodiments 1-14 or 29-44 or the FDFT1 inhibitor or pharmaceutically acceptable salt thereof for use according to any one of embodiments 15-44, wherein the FDFT1 inhibitor prevents sexual development of the parasite.
46. The method of any one of embodiments 1-14 or 29-45 or the FDFT1 inhibitor or pharmaceutically acceptable salt thereof for use according to any one of embodiments 15-45, wherein the FDFT1 inhibitor reduces the reproduction of the parasite.
47. The method of any one of embodiments 1-14 or 29-46 or the FDFT1 inhibitor or pharmaceutically acceptable salt thereof for use according to any one of embodiments 15-46, wherein the method comprises administering to patient the FDFT1 inhibitor or pharmaceutically acceptable salt thereof at a dose of 0.0001 mg/kg to 100 mg/kg.
48. The method of any one of embodiments 1-14 or 29-47 or the FDFT1 inhibitor or pharmaceutically acceptable salt thereof for use according to any one of embodiments 15-47, wherein the method comprises administering to patient the FDFT1 inhibitor or pharmaceutically acceptable salt thereof at a dose of 0.01 mg/kg to 5 mg/kg.
49. The method of any one of embodiments 1-14 or 29-48 or the FDFT1 inhibitor or pharmaceutically acceptable salt thereof for use according to any one of embodiments 15-48, wherein the method comprises administering a single daily dose of the FDFT1 inhibitor or pharmaceutically acceptable salt thereof to the patient.
50. The method of any one of embodiments 1-14 or 29-49 or the FDFT1 inhibitor or pharmaceutically acceptable salt thereof for use according to any one of embodiments 15-49, wherein the method comprises administering a single daily dose of 10 mg to 200 mg of the FDFT1 inhibitor or pharmaceutically acceptable salt thereof to the patient.
51. The method of any one of embodiments 1-14 or 29-50 or the FDFT1 inhibitor or pharmaceutically acceptable salt thereof for use according to any one of embodiments 15-50, wherein the method comprises administering a single daily dose of 25 mg to 100 mg of the FDFT1 inhibitor or pharmaceutically acceptable salt thereof to the patient.
52. The method of any one of embodiments 1-14 or 29-51 or the FDFT1 inhibitor or pharmaceutically acceptable salt thereof for use according to any one of embodiments 15-51, wherein the method comprises administering a single daily dose of 25 mg, 50 mg or 100 mg of the FDFT1 inhibitor or pharmaceutically acceptable salt thereof to the patient.
53. The method of any one of embodiments 1-14 or 29-52 or the FDFT1 inhibitor or pharmaceutically acceptable salt thereof for use according to any one of embodiments 15-52, wherein the method comprises administering a single daily dose of 25 mg, 50 mg or 100 mg of lapaquistat or a pharmaceutically acceptable salt thereof to the patient.
54. The method of any one of embodiments 1-14 or 29-52 or the FDFT1 inhibitor or pharmaceutically acceptable salt thereof for use according to any one of embodiments 15-52, wherein the method comprises administering a single daily dose of 25 mg of lapaquistat or a pharmaceutically acceptable salt thereof to the patient.
55. The method of any one of embodiments 1-14 or 29-52 or the FDFT1 inhibitor or pharmaceutically acceptable salt thereof for use according to any one of embodiments 15-52, wherein the method comprises administering a single daily dose of 50 mg of lapaquistat or a pharmaceutically acceptable salt thereof to the patient.
56. The method of any one of embodiments 1-14 or 29-52 or the FDFT1 inhibitor or pharmaceutically acceptable salt thereof for use according to any one of embodiments 15-52, wherein the method comprises administering a single daily dose of 100 mg of lapaquistat or a pharmaceutically acceptable salt thereof to the patient.
57. The method of any one of embodiments 1-14 or 29-56 or the FDFT1 inhibitor or pharmaceutically acceptable salt thereof for use according to any one of embodiments 15-56, wherein the method further comprises determining whether a patient has a parasitic infection.
58. The method of any one of embodiments 1-14 or 29-56 or the FDFT1 inhibitor or pharmaceutically acceptable salt thereof for use according to any one of embodiments 15-56, wherein the method further comprises determining whether a patient has a viral infection.
59. The method of any one of embodiments 1-14 or 29-58 or the FDFT1 inhibitor or pharmaceutically acceptable salt thereof for use according to any one of embodiments 15-58, wherein the parasitic infection is an infection by an intracellular parasite which is incapable of endogenous cholesterol biosynthesis.
60. The method of any one of embodiments 1-14 or 29-59 or the FDFT1 inhibitor or pharmaceutically acceptable salt thereof for use according to any one of embodiments 15-59, wherein the parasitic infection is an infection by an intracellular parasite which is incapable of endogenous squalene synthesis.
61. The method of any one of embodiments 1-14 or 29-60 or the FDFT1 inhibitor or pharmaceutically acceptable salt thereof for use according to any one of embodiments 15-60, wherein the parasitic infection is an infection by an intracellular parasite which does not express an endogenous squalene synthase.
62. The method of any one of embodiments 1-14 or 29-61 or the FDFT1 inhibitor or pharmaceutically acceptable salt thereof for use according to any one of embodiments 15-61, wherein the parasitic infection is an infection by an intracellular parasite which is an obligate intracellular parasite.
63. The method of any one of embodiments 1-14 or 29-62 or the FDFT1 inhibitor or pharmaceutically acceptable salt thereof for use according to any one of embodiments 15-62, wherein the parasitic infection is an infection by an intracellular parasite which is in the order of Eucoccidiorida, preferably in the suborder of Eimeriorina.
64. The method of any one of embodiments 1-14 or 29-63 or the FDFT1 inhibitor or pharmaceutically acceptable salt thereof for use according to any one of embodiments 15-63, wherein the parasitic infection is an infection by a parasite which is a Cryptosporidium spp.
65. The method of any one of embodiments 1-14 or 29-64 or the FDFT1 inhibitor or pharmaceutically acceptable salt thereof for use according to any one of embodiments 15-64, wherein the FDFT1 inhibitor is administered simultaneously, sequentially or separately with one or more other drugs which inhibit cholesterol biosynthesis and/or which reduce the amount of cholesterol in the blood of the subject.
66. The method or FDFT1 inhibitor or pharmaceutically acceptable salt thereof for use according to embodiment 65, wherein the one or more other drugs which inhibit cholesterol biosynthesis and/or which reduce the amount of cholesterol in the blood of the subject is a statin.
67. The method or FDFT1 inhibitor or pharmaceutically acceptable salt thereof for use according to embodiment 66, wherein the statin is selected from one or more of lovastatin, atorvastatin, fluvastatin, pitavastatin, pravastatin, rosuvastatin, and simvastatin, preferably wherein the statin is lovastatin or atorvastatin.
68. The method of any one of embodiments 1-14 or 29-67 or the FDFT1 inhibitor or pharmaceutically acceptable salt thereof for use according to any one of embodiments 15-67, wherein the FDFT1 inhibitor is lapaquistat and wherein the lapaquistat is administered in combination with lovastatin or atorvastatin.
69. Use of a therapeutically or prophylactically effective amount of a farnesyl-diphosphate farnesyltransferase 1 (FDFT1) inhibitor, or a pharmaceutically acceptable salt thereof in a method of treating or preventing a parasitic infection.
70. Use of a therapeutically or prophylactically effective amount of a farnesyl-diphosphate farnesyltransferase 1 (FDFT1) inhibitor, or a pharmaceutically acceptable salt thereof in a method of treating or preventing a Cryptosporidium infection.
71. Use of a therapeutically or prophylactically effective amount of a farnesyl-diphosphate farnesyltransferase 1 (FDFT1) inhibitor, or a pharmaceutically acceptable salt thereof in a method of treating or preventing a viral infection.
72. Use of a therapeutically or prophylactically effective amount of a farnesyl-diphosphate farnesyltransferase 1 (FDFT1) inhibitor, or a pharmaceutically acceptable salt thereof in a method of treating or preventing a rotavirus infection.
73. Use of a farnesyl-diphosphate farnesyltransferase 1 (FDFT1) inhibitor, or a pharmaceutically acceptable salt thereof, in a method of treating or preventing an infection.
74. The use of a farnesyl-diphosphate farnesyltransferase 1 (FDFT1) inhibitor, or a pharmaceutically acceptable salt thereof, according to embodiment 73, wherein the infection is an infection by a pathogen which is incapable of endogenous cholesterol biosynthesis.
75. The use of a farnesyl-diphosphate farnesyltransferase 1 (FDFT1) inhibitor, or a pharmaceutically acceptable salt thereof, according to embodiment 73 or 74, wherein the infection is an infection by a pathogen which is incapable of endogenous squalene synthesis.
76. The use of a farnesyl-diphosphate farnesyltransferase 1 (FDFT1) inhibitor, or a pharmaceutically acceptable salt thereof, according to any one of embodiments 73 to 75, wherein, the infection is an infection by a pathogen which does not express an endogenous squalene synthase.
77. The use of a farnesyl-diphosphate farnesyltransferase 1 (FDFT1) inhibitor, or a pharmaceutically acceptable salt thereof, according to any one of embodiments 73 to 76, wherein the infection is a parasitic, viral or bacterial infection.
78. The use of a farnesyl-diphosphate farnesyltransferase 1 (FDFT1) inhibitor, or a pharmaceutically acceptable salt thereof, according to embodiment 77, wherein the infection is a Cryptosporidium infection.
79. The use of a farnesyl-diphosphate farnesyltransferase 1 (FDFT1) inhibitor, or a pharmaceutically acceptable salt thereof, according to embodiment 77, wherein the infection is a rotavirus infection.
80. The use of a farnesyl-diphosphate farnesyltransferase 1 (FDFT1) inhibitor, or a pharmaceutically acceptable salt thereof, according to embodiment 77, wherein the infection is a Salmonella infection.
81. A pharmaceutical composition comprising a farnesyl-diphosphate farnesyltransferase 1 (FDFT1) inhibitor, or a pharmaceutically acceptable salt thereof.
82. The pharmaceutical composition of embodiment 81, wherein the FDFT1 inhibitor is lapaquistat or YM53601.
83. The pharmaceutical composition of embodiment 81 or 82, wherein the composition further comprises a drug which inhibits cholesterol biosynthesis and/or which reduces the amount of cholesterol in the blood of the subject
84. The pharmaceutical composition of embodiment 81 or 82, wherein the composition further comprises a statin.
85. The pharmaceutical composition of embodiment 84, wherein the statin is selected from the group consisting of lovastatin, atorvastatin, fluvastatin, pitavastatin, pravastatin, rosuvastatin, and simvastatin.
86. The pharmaceutical composition of embodiment 85, wherein the statin is selected from the group consisting of lovastatin and atorvastatin.
87. The pharmaceutical composition of any one of embodiments 81 to 86, wherein the composition comprises:
   a. an FDFT1 inhibitor and a statin;
   b. Lapaquistat and a statin;
   c. Lapaquistat and lovastatin;
   d. Lapaquistat and atorvastatin;
   e. YM53601 and a statin;
   f. YM53601 and lovastatin; or
   g. YM53601 and atorvastatin.
88. The pharmaceutical composition of any one of embodiments 81 to 87 for use in the treatment or prevention of an intracellular infection.
89. The pharmaceutical composition for use according to embodiment 88, wherein the infection is an infection by an intracellular pathogen which is incapable of endogenous cholesterol biosynthesis.
90. The pharmaceutical composition for use according to embodiment 88 or 89, wherein the infection is an infection by an intracellular pathogen which is incapable of endogenous squalene synthesis.
91. The pharmaceutical composition for use according to embodiment 88, 89 or 90, wherein the infection is an infection by an intracellular pathogen which does not express an endogenous squalene synthase.
92. The pharmaceutical composition for use according to any one of embodiments 88 to 91, wherein the infection is an intracellular parasitic, viral or bacterial invention.
93. The pharmaceutical composition for use according to any one of embodiments 88 to 92, wherein the infection is an intracellular parasitic infection.
94. The pharmaceutical composition for use according to any one of embodiments 88 to 93, wherein the infection is a Cryptosporidium infection, a rotavirus infection, or a Salmonella infection.
95. The pharmaceutical composition for use according to any one of embodiments 88 to 94, wherein the infection is a Cryptosporidium infection.
96. The pharmaceutical composition of any one of embodiments 81 to 87, wherein the composition further comprises one or more other drugs used in the treatment or prophylaxis of a parasitic infection, such as a Cryptosporidium infection.
97. The pharmaceutical composition of any one of embodiments 81 to 87, wherein the composition further comprises nitazoxanide, paromomycin or halofuginone.
98. The pharmaceutical composition of any one of embodiments 81 to 87, wherein the composition further comprises nitazoxanide.
99. The pharmaceutical composition of any one of embodiments 96 to 98, wherein the composition comprises:
   a. a FDFT1 inhibitor and one or more of nitazoxanide, paromomycin or halofuginone
   b. Lapaquistat and one or more of nitazoxanide, paromomycin or halofuginone;
   c. YM53601 and one or more of nitazoxanide, paromomycin or halofuginone;
   d. Lapaquistat and nitazoxanide;
   e. YM53601 and nitazoxanide;
   f. a FDFT1 inhibitor, a statin and one or more of nitazoxanide, paromomycin or halofuginone;
   g. Lapaquistat, a statin and one or more of nitazoxanide, paromomycin or halofuginone;
   h. Lapaquistat, lovastatin and one or more of nitazoxanide, paromomycin or halofuginone;
   i. Lapaquistat, atorvastatin and one or more of nitazoxanide, paromomycin or halofuginone;
   j. YM53601, a statin and one or more of nitazoxanide, paromomycin or halofuginone;
   k. YM53601, lovastatin and one of nitazoxanide, paromomycin or halofuginone;
   l. YM53601, atorvastatin and one of nitazoxanide, paromomycin or halofuginone;
   m. Lapaquistat, a statin and nitazoxanide;
   n. Lapaquistat, lovastatin and nitazoxanide;
   o. Lapaquistat, atorvastatin and nitazoxanide;
   p. YM53601, a statin and nitazoxanide;
   q. YM53601, lovastatin and nitazoxanide;
   r. YM53601, atorvastatin and nitazoxanide.
100. The pharmaceutical composition of any one of embodiments 96 to 99 for use in the treatment or prevention of an intracellular infection.
101. The pharmaceutical composition for use according to embodiment 100, wherein the infection is an infection by an intracellular pathogen which is incapable of endogenous cholesterol biosynthesis.
102. The pharmaceutical composition for use according to embodiment 100 or 101, wherein the infection is an infection by an intracellular pathogen which is incapable of endogenous squalene synthesis.
103. The pharmaceutical composition for use according to embodiment 100, 101 or 102, wherein the infection is an infection by an intracellular pathogen which does not express an endogenous squalene synthase.
104. The pharmaceutical composition for use according to any one of embodiments 100 to 103, wherein the infection is an intracellular parasitic infection.
105. The pharmaceutical composition for use according to any one of embodiments 100 to 104, wherein the infection is a Cryptosporidium infection.
106. The combination of a farnesyl-diphosphate farnesyltransferase 1 (FDFT1) inhibitor, or a pharmaceutically acceptable salt thereof, and:
   a. a drug which inhibits cholesterol biosynthesis and/or which reduces the amount of cholesterol in the blood of the subject; and/or
   b. one or more other drugs used in the treatment or prophylaxis of a parasitic infection.
107. The combination of embodiment 106, wherein the FDFT1 inhibitor is lapaquistat or YM53601.
108. The combination of embodiment 106 or 107, wherein the composition further comprises a drug which inhibits cholesterol biosynthesis and/or which reduces the amount of cholesterol in the blood of the subject.
109. The combination of any one of embodiments 106 to 108, wherein the drug which inhibits cholesterol biosynthesis and/or which reduces the amount of cholesterol in the blood of the subject is a statin.
110. The combination of embodiment 109, wherein the statin is selected from the group consisting of lovastatin, atorvastatin, fluvastatin, pitavastatin, pravastatin, rosuvastatin, and simvastatin.
111. The combination of embodiment 109, wherein the statin is selected from the group consisting of lovastatin and atorvastatin.
112. The combination of any one of embodiments 106 to 111, wherein the composition comprises:
   a. a FDFT1 inhibitor and a statin;
   b. Lapaquistat and a statin;
   c. Lapaquistat and lovastatin;
   d. Lapaquistat and atorvastatin;
   e. YM53601 and a statin;
   f. YM53601 and lovastatin; or
   g. YM53601 and atorvastatin.
113. The combination of any one of embodiments 106 to 112 for use in the treatment or prevention of an intracellular infection.
114. The combination for use according to embodiment 113, wherein the infection is an infection by an intracellular pathogen which is incapable of endogenous cholesterol biosynthesis.
115. The combination for use according to embodiment 113 or 114, wherein the infection is an infection by an intracellular pathogen which is incapable of endogenous squalene synthesis.
116. The combination for use according to any one of embodiments 113 to 115, wherein the infection is an infection by an intracellular pathogen which does not express an endogenous squalene synthase.
117. The combination for use according to any one of embodiments 113 to 116, wherein the infection is an intracellular parasitic, viral or bacterial invention.
118. The combination for use according to any one of embodiments 113 to 117, wherein the infection is an intracellular parasitic infection.
119. The combination for use according to any one of embodiments 113 to 118, wherein the infection is a Cryptosporidium infection, a rotavirus infection, or a Salmonella infection.
120. The combination for use according to any one of embodiments 113 to 119, wherein the infection is a Cryptosporidium infection.
121. The combination of any one of embodiments 106 to 112, wherein the combination further comprises one or more other drugs used in the treatment or prophylaxis of a parasitic infection, such as a Cryptosporidium infection.
122. The combination of 112, wherein the one or more other drugs used in the treatment or prophylaxis of a parasitic infection is nitazoxanide, paromomycin or halofuginone.
123. The combination embodiment 112, wherein the one or more other drugs used in the treatment or prophylaxis of a parasitic infection is nitazoxanide.
124. The combination of any one of embodiments 106 to 112, wherein the combination comprises:
   a. a FDFT1 inhibitor and one or more of nitazoxanide, paromomycin or halofuginone
   b. Lapaquistat and one or more of nitazoxanide, paromomycin or halofuginone;
   c. YM53601 and one or more of nitazoxanide, paromomycin or halofuginone;
   d. Lapaquistat and nitazoxanide;
   e. YM53601 and nitazoxanide.
   f. a FDFT1 inhibitor, a statin and one or more of nitazoxanide, paromomycin or halofuginone;
   g. Lapaquistat, a statin and one or more of nitazoxanide, paromomycin or halofuginone;
   h. Lapaquistat, lovastatin and one or more of nitazoxanide, paromomycin or halofuginone;
   i. Lapaquistat, atorvastatin and one or more of nitazoxanide, paromomycin or halofuginone;
   j. YM53601, a statin and one or more of nitazoxanide, paromomycin or halofuginone;
   k. YM53601, lovastatin and one or more of nitazoxanide, paromomycin or halofuginone;
   l. YM53601, atorvastatin and one or more of nitazoxanide, paromomycin or halofuginone;
   m. Lapaquistat, a statin and nitazoxanide;
   n. Lapaquistat, lovastatin and nitazoxanide;
   o. Lapaquistat, atorvastatin and nitazoxanide;
   p. YM53601, a statin and nitazoxanide;
   q. YM53601, lovastatin and nitazoxanide;
   r. YM53601, atorvastatin and nitazoxanide;
125. The combination of any one of embodiments 121 to 124 for use in the treatment or prevention of an intracellular infection.
126. The combination for use according to embodiment 125, wherein the infection is an infection by an intracellular pathogen which is incapable of endogenous cholesterol biosynthesis.
127. The combination for use according to embodiment 125 or 126, wherein the infection is an infection by an intracellular pathogen which is incapable of endogenous squalene synthesis.
128. The combination for use according to any one of embodiments 125 to 127, wherein the infection is an infection by an intracellular pathogen which does not express an endogenous squalene synthase.
129. The combination for use according to any one of embodiments 125 to 128, wherein the infection is an intracellular parasitic infection.
130. The combination for use according to any one of embodiments 125 to 129, wherein the infection is a Cryptosporidium infection.
131. The combination of any one of embodiments 106 to 112 or 121 to 124, or the combination for use according to any one of embodiments 113 to 120 or 125 to 130, wherein the components of the combination are for separate, simultaneous or sequential administration.
132. A kit comprising the components of the combination of any one of embodiments 106 to 112 or 121 to 124.
133. The kit of embodiments 132, wherein the components of the kit are disposed separately in separate containers.
134. The kit of embodiment 132 or 133, further comprising instructions for use.
135. A statin for use in a method of treating or preventing an infection, wherein the statin is administered simultaneously, sequentially or separately with a FDFT1 inhibitor, or a pharmaceutically acceptable salt thereof.
136. The statin for use according to embodiment 135, further characterised by the features of any one or more of embodiments 1 to 134.
137. The statin for use according to embodiment 135 or embodiment 136, wherein the statin is lovastatin or atorvastatin.
138. The statin for use according to any one of embodiments 135 to 137, wherein the FDFT1 inhibitor is lapaquistat.
139. The statin for use according to any one of embodiments 135 to 138, wherein the infection is an infection by an intracellular parasite which is incapable of endogenous cholesterol biosynthesis, preferably wherein the parasite is a Cryptosporidium spp.
135. A statin for use in a method of treating or preventing a Cryptosporidium infection, wherein the statin is lovastatin or atorvastatin and is administered simultaneously, sequentially or separately with lapaquistat.

### REFERENCES

[1] Use-case scenarios for an anti-Cryptosporidium therapeutic, Ashigbie P G et al., PLoS Neglected Tropical Disease 2021
[2] A review of the global burden, novel diagnostics, therapeutics, and vaccine targets for Cryptosporidium. Checkley W et al., Lancet Infectious Diseases 2015
[3] Ugawa, T., Kakuta, H., Moritani, H., Matsuda, K., Ishihara, T., Yamaguchi, M., Naganuma, S., Iizumi, Y. and Shikama, H. (2000), YM-53601, a novel squalene synthase inhibitor, reduces plasma cholesterol and triglyceride levels in several animal species. British Journal of Pharmacology, 131: 63-70.
[4] Amin, D., S. A. Cornell, S. K. Gustafson, S. J. Needle, J. W. Ullrich, G. E. Bilder, and M. H. Perrone. Bisphosphonates used for the treatment of bone disorders inhibit squalene synthase and cholesterol biosynthesis. J. Lipid Res. 1992. 33: 1657-1663.
[5] Synthesis of Novel 4,1-Benzoxazepine Derivatives as Squalene Synthase Inhibitors and Their Inhibition of Cholesterol Synthesis; Takashi Miki, Masakuni Kori, Hiroshi Mabuchi, Ryu-ichi Tozawa, Tomoyuki Nishimoto, Yasuo Sugiyama, Koichiro Teshima, and Hidefumi Yukimasa Journal of Medicinal Chemistry 2002 45 (20), 4571-4580
[6] Inhibition of cholesterol synthesis by squalene synthase inhibitors does not induce myotoxicity in vitro. Flint O P et al., Toxicology and Applied Pharmacology 1997
[7] Lipid-lowering properties of TAK-475, a squalene synthase inhibitor, in vivo and in vitro. Nishimoto T et al., British Journal of Pharmacology 2003
[8] Development of a Squalene Synthase Inhibitor for the Treatment of Hypercholesterolemia. Stein E A et al., Circulation 2011

1. A farnesyl-diphosphate farnesyltransferase 1 (FDFT1) inhibitor, or a pharmaceutically acceptable salt thereof for use in a method of treating or preventing a parasitic infection.

2. The FDFT1 inhibitor or pharmaceutically acceptable salt thereof for use according to claim 1, wherein the parasitic infection is an infection by an intracellular parasite which is incapable of endogenous cholesterol biosynthesis.

3. The FDFT1 inhibitor or pharmaceutically acceptable salt thereof for use according to claim 1 or claim 2, wherein the parasitic infection is an infection by an intracellular parasite which is incapable of endogenous squalene synthesis.

4. The FDFT1 inhibitor or pharmaceutically acceptable salt thereof for use according to any preceding claim, wherein the parasitic infection is an infection by an intracellular parasite which does not express an endogenous squalene synthase.

5. The FDFT1 inhibitor or pharmaceutically acceptable salt thereof for use according to any preceding claim, wherein the parasitic infection is an infection by an intracellular parasite which is an obligate intracellular parasite.

6. The FDFT1 inhibitor or pharmaceutically acceptable salt thereof for use according to any preceding claim, wherein the parasitic infection is an infection by a parasite which is in the order of Eucoccidiorida, preferably in the suborder of Eimeriorina.

7. The FDFT1 inhibitor or pharmaceutically acceptable salt thereof for use according to any preceding claim, wherein the parasitic infection is an infection by a Cryptosporidium spp.

8. A farnesyl-diphosphate farnesyltransferase 1 (FDFT1) inhibitor, or a pharmaceutically acceptable salt thereof for use in a method of treating or preventing a Cryptosporidium infection.

9. A farnesyl-diphosphate farnesyltransferase 1 (FDFT1) inhibitor, or a pharmaceutically acceptable salt thereof for use in a method of treating or preventing a viral infection.

10. A farnesyl-diphosphate farnesyltransferase 1 (FDFT1) inhibitor, or a pharmaceutically acceptable salt thereof for use in a method of treating or preventing a rotavirus infection.

11. A farnesyl-diphosphate farnesyltransferase 1 (FDFT1) inhibitor, or a pharmaceutically acceptable salt thereof for use in a method of treating or preventing a bacterial infection.

12. A farnesyl-diphosphate farnesyltransferase 1 (FDFT1) inhibitor, or a pharmaceutically acceptable salt thereof for use in a method of treating or preventing a Salmonella infection.

13. The FDFT1 inhibitor or pharmaceutically acceptable salt thereof for use according to any preceding claim, wherein the FDFT1 inhibitor has the following structure:

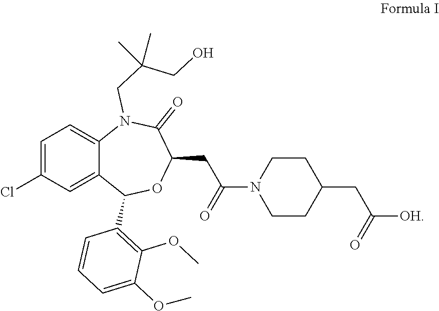 [SMILES: COc1cccc([C@H]2O[C@H](CC(=O)N3CCC(CC(=O)O)CC3)C(=O)N(CC(C)(C)CO)c3ccc(Cl)cc32)c1OC]

14. The FDFT1 inhibitor or pharmaceutically acceptable salt thereof for use according to any preceding claim, wherein the FDFT1 inhibitor is lapaquistat.

15. The FDFT1 inhibitor or pharmaceutically acceptable salt thereof for use according to any one of claims 1-12, wherein the FDFT1 inhibitor is YM-53601, or a pharmaceutically acceptable salt thereof.

16. The FDFT1 inhibitor or pharmaceutically acceptable salt thereof for use according to any one of claims 1-12, wherein the FDFT1 inhibitor has the following structure:

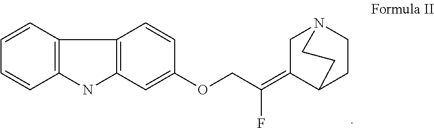 [SMILES: F/C(COc1ccc2c(c1)[nH]c1ccccc12)=C1/CN2CCC1CC2]

17. The FDFT1 inhibitor or pharmaceutically acceptable salt thereof for use according to any preceding claim, wherein the FDFT1 inhibitor is administered simultaneously, sequentially or separately with one or more other drugs used in the treatment or prophylaxis of the parasitic infection or Cryptosporidium infection.

18. The FDFT1 inhibitor or pharmaceutically acceptable salt thereof for use according to claim 17, wherein the one or more other drugs is formulated in a combined preparation with the FDFT1 inhibitor or pharmaceutically acceptable salt thereof.

19. The FDFT1 inhibitor or pharmaceutically acceptable salt thereof for use according to claim 17 or claim 18, wherein the one or more other drugs comprises nitazoxanide, paromomycin or halofuginone.

20. The FDFT1 inhibitor or pharmaceutically acceptable salt thereof for use according to any one of claims 17-19, wherein the one or more other drugs comprises nitazoxanide.

21. The FDFT1 inhibitor or pharmaceutically acceptable salt thereof for use according to any preceding claim, wherein the FDFT1 inhibitor is administered simultaneously, sequentially or separately with one or more other drugs which inhibit cholesterol biosynthesis and/or which reduce the amount of cholesterol in the blood of the subject.

22. The FDFT1 inhibitor or pharmaceutically acceptable salt thereof for use according to claim 21, wherein the one or more other drugs which inhibit cholesterol biosynthesis and/or which reduce the amount of cholesterol in the blood of the subject is a statin.

23. The FDFT1 inhibitor or pharmaceutically acceptable salt thereof for use according to claim 22, wherein the statin is selected from one or more of lovastatin, atorvastatin, fluvastatin, pitavastatin, pravastatin, rosuvastatin, and simvastatin, preferably wherein the statin is lovastatin or atorvastatin.

24. The FDFT1 inhibitor or pharmaceutically acceptable salt thereof for use according to any preceding claim, wherein the FDFT1 inhibitor is lapaquistat and wherein the lapaquistat is administered simultaneously, sequentially or separately with lovastatin or atorvastatin.

25. The FDFT1 inhibitor or pharmaceutically acceptable salt thereof for use according to any preceding claim, wherein the FDFT1 inhibitor is administered to the patient orally, intravenously, intramuscularly, intrathecally, subcutaneously, sublingually, buccally, rectally, vaginally, ocularly, oticly, nasally, by inhalation, by nebulization, cutaneously, topically or transdermally.

26. The FDFT1 inhibitor or pharmaceutically acceptable salt thereof for use according to any preceding claim, wherein the subject is a human.

27. The FDFT1 inhibitor or pharmaceutically acceptable salt thereof for use according to any one of claims 1-25, wherein the subject is a cow (cattle), a sheep, a goat, a cat, a dog, a pig (swine), a horse or a rabbit.

28. The FDFT1 inhibitor or pharmaceutically acceptable salt thereof for use according to any preceding claim, wherein the FDFT1 inhibitor is administered in a dose sufficient to achieve at least 50%, at least 60%, at least 70%, at least 80%, at least 90%, at least 95% or at least 99% inhibition of FDFT1 activity.

29. The FDFT1 inhibitor or pharmaceutically acceptable salt thereof for use according to any preceding claim, wherein the FDFT1 inhibitor is administered in a dose sufficient to achieve at least 50%, at least 60%, at least 70%, at least 80%, at least 90%, at least 95% or at least 99% inhibition of squalene production in a target cell.

30. The FDFT1 inhibitor or pharmaceutically acceptable salt thereof for use according to any preceding claim, wherein the FDFT1 inhibitor prevents sexual development of the parasite.

31. The FDFT1 inhibitor or pharmaceutically acceptable salt thereof for use according to any preceding claim, wherein the FDFT1 inhibitor reduces the reproduction of the parasite.

32. The FDFT1 inhibitor or pharmaceutically acceptable salt thereof for use according to any preceding claim, wherein the method comprises administering to patient the FDFT1 inhibitor or pharmaceutically acceptable salt thereof at a dose of 0.0001 mg/kg to 100 mg/kg.

33. The FDFT1 inhibitor or pharmaceutically acceptable salt thereof for use according to any preceding claim, wherein the method comprises administering a single daily dose of 25 mg to 100 mg of the FDFT1 inhibitor or pharmaceutically acceptable salt thereof to the patient.

34. Use of a therapeutically or prophylactically effective amount of a farnesyl-diphosphate farnesyltransferase 1 (FDFT1) inhibitor, or a pharmaceutically acceptable salt thereof in a method of treating or preventing a parasitic infection.

35. Use of a therapeutically or prophylactically effective amount of a farnesyl-diphosphate farnesyltransferase 1 (FDFT1) inhibitor, or a pharmaceutically acceptable salt thereof in a method of treating or preventing a Cryptosporidium infection.
